SEQUENCE LISTING (70 sequences)

SEQ ID NO: 1 - DNA, 2649 bp, Homo sapiens
  CDS (1)..(2649)
atggctgtgcgctctcgccgcccgtggatgagcgtggcattagggctggtgctgggcttcaccgccgcgtcctggctcatcgcccccagggtggcggagctgagcgagaggaagagacgtggctccagcctctgctcctactacggtcgctctgctgctggcccccgcgccggcgctcagcagccgctcccccagccccagtcccgaccacggcaggagcagtcgccgccccccgcgcgccaggatctccaggggccaccgctgcccgaggcagcacccgggatcaccagttttcgaagcagcccctggcagcagccacctccgctgcagcagcggcggcgaggacgcgagcctgagggcgcgacggggcttcccggtgctccagcggccgagggggagcccgaggaggaggacgggggcgcggctgggcagcggagagacggccggccggggagtagccacaacggcagcggggacgggggcgctgccgccccgagcgcccgaccccgggacttcctgtacgtgggggtgatgaccgcgcagaagtacctgggcagccgcgcgctggccgcgcagcggacctgggcgcgtttcatcccgggccgcgtggagttcttttccagccagcagccccccaacgccggccagcccccgccacccctgcctgtcatcgcgctaccgggtgtggacgactcctatcctccccagaaaaagtccttcatgatgatcaagtacatgcacgaccactacctggacaagtatgagtggttcatgcgcgccgacgacgatgtctacatcaaaggtgataaattagaagagtttcttagatcgctaaacagcagtaagcctctctacctgggacagactggcctggggaatattgaagagcttggaaagctgggactggagcctggggaaaacttctgtatgggaggacctggcatgatctttagccgagaagttctcaggaggatggtgccacatattggtgaatgccttagagaaatgtacacgactcatgaggatgtggaagtaggaagatgcgttcgccgttttggtgggactcagtgtgtctggtcttacgagatgcaacaactgttccatgaaaattatgaacacaatcggaagggttacatccaagaccttcacaatagcaaaatccatgcagccataacacttcatcccaacaaaaggcctgcataccaatacaggctgcataattacatgctcagccgcaaaatttctgaacttcgctaccgcaccatccagctccacagggaaagtgccctgatgagcaagctcagtaacacagaagtgagcaaagaggaccagcagctgggagtgataccttctttcaaccacttccagcctcgggagagaaatgaagtgatagaatgggagttcctgacagggaagcttctatactcagcagctgagaaccagccccctcgacagagcctcagtagcattttaagaacagcactggatgataccgtcctacaggtgatggagatgatcaatgagaatgccaagagcagaggacggctcattgacttcaaggaaattcagtatggctaccgcagagttaaccccatgcacggggtggagtacattttggatttactccttttatacaaaagacacaagggaaggaaactgactgtgccagtgagacgtcatgcctatcttcagcagttgttcagcaagcctttcttcagagagaccgaagagctagatgtcaacagtcttgtggagagtattaacagtgaaactcagtcattctcctttatatctaattctttaaagatattatcttcttttcaaggtgccaaagaaatgggagggcacaatgaaaagaaagtacacattctcgttcctctcatcggaaggtatgacattttcttgagattcatggagaactttgaaaacatgtgtcttatcccaaagcagaatgtaaagttggtcattatccttttcagtagggattctggccaagactccagcaagcatattgagctgataaaagggtaccagaacaagtaccccaaagcagaaatgaccctgatcccaatgaagggagagttttccagaggtcttggtcttgaaatggcttctgcccagtttgacaatgacactttgctgctattttgtgatgttgacttgatcttcagagaagattttctccaacgatgtagagacaatacaattcagggacaacaggtgtactatcccatcatctttagccagtatgacccaaaggtaacaaacgggggaaatcctcccactgatggttacttcatattctcaaaaaagactggattttggagagactatggatatggcatcacctgtatttacaaaagtgatcttctaggtgcaggtggatttgatacctcaatacaaggctggggactagaagatgtagatctctacaataaagtcattctatctggcttaaggccattcagaagccaagaagtaggagtggtgcatattttccatccagttcattgtgatcctaacttggaccctaagcagtataagatgtgcttaggatccaaggcaagtactttcgcctcaaccatgcaactggctgaactctggcttgaaaaacatttaggtgtcaggtacaatcgaactctctcctga

SEQ ID NO: 2 - protein, 882 aa, Homo sapiens [sequence omitted]

SEQ ID NO: 3 - DNA, 2328 bp, Homo sapiens
  CDS (1)..(2328)
atgcgggcatcgctgctgctgtcggtgctgcggcccgcagggcccgtggccgtgggcatctccctgggcttcaccctgagcctgctcagcgtcacctgggtggaggagccgtgcggcccaggcccgccccaacctggagactctgagctgccgccgcgcggcaacaccaacgcggcgcgccggcccaactcggtgcagcccggagcggagcgcgagaagcccggggccggcgaaggcgccggggagaattgggagccgcgcgtcttgccctaccaccctgcacagcccggccaggccgccaaaaaggccgtcaggacccgctacatcagcacggagctgggcatcaggcagaggctgctggtggcggtgctgacctctcagaccacgctgcccacgctgggcgtggccgtgaaccgcacgctggggcaccggctggagcgtgtggtgttcctgacgggcgcacggggccgccgggccccacctggcatggcagtggtgacgctgggcgaggagcgacccattggacacctgcacctggcgctgcgccacctgctggagcagcacggcgacgactttgactggttcttcctggtgcctgacaccacctacaccgaggcgcacggcctggcacgcctaactggccacctcagcctggcctccgccgcccacctgtacctgggccggccccaggacttcatcggcggagagcccacccccggccgctactgccacggaggctttggggtgctgctgtcgcgcatgctgctgcaacaactgcgcccccacctggaaggctgccgcaacgacatcgtcagtgcgcgccctgacgagtggctgggtcgctgcattctcgatgccaccggggtgggctgcactggtgaccacgagggggtgcactatagccatctggagctgagccctggggagccagtgcaggagggggaccctcatttccgaagtgccctgacagcccaccctgtgcgtgaccctgtgcacatgtaccagctgcacaaagctttcgcccgagctgaactggaacgcacgtaccaggagatccaggagttacagtgggagatccagaataccagccatctggccgttgatggggaccgggcagctgcttggcccgtgggtattccagcaccatcccgcccggcctcccgctttgaggtgctgcgctgggactacttcacggagcagcacgctttctcctgcgccgatggctcaccccgctgcccactgcgtggggctgaccgggctgatgtggccgatgttctggggacagctctagaggagctgaaccgccgctaccacccggccttgcggctccagaagcagcagctggtgaatggctaccgacgctttgatccggcccggggtatggaatacacgctggacttgcagctggaggcactgaccccccagggaggccgccggcccctcactcgccgagtgcagctgctccggccgctgagccgcgtggagatcttgcctgtgccctatgtcactgaggcctcacgtctcactgtgctgctgcctctagctgcggctgagcgtgacctggcccctggcttcttggaggcctttgccactgcagcactggagcctggtgatgctgcggcagccctgaccctgctgctactgtatgagccgcgccaggcccagcgcgtggcccatgcagatgtcttcgcacctgtcaaggcccacgtggcagagctggagcggcgtttccccggtgcccgggtgccatggctcagtgtgcagacagccgcaccctcaccactgcgcctcatggatctactctccaagaagcacccgctggacacactgttcctgctggccgggccagacacggtgctcacgcctgacttcctgaaccgctgccgcatgcatgccatctccggctggcaggccttctttcccatgcatttccaagccttccacccagctgtggccccaccacaagggcctgggcccccagagctgggccgtgacactggccgctttgatcgccaggcagccagcgaggcctgcttctacaactccgactacgtggcagcccgtgggcgcctggcggcagcctcagaacaagaagaggagctgctggagagcctggatgtgtacgagctgttcctccacttctccagtctgcatgtgctgcgggcggtggagccggcgctgctgcagcgctaccgggcccagacgtgcagcgcgaggctcagtgaggacctgtaccaccgctgcctccagagcgtgcttgagggcctcggctcccgaacccagctggccatgctactctttgaacaggagcagggcaacagcacctga

SEQ ID NO: 4 - protein, 775 aa, Homo sapiens
MRASLLLSVLRPAGPVAVGISLGFTLSLLSVTWVEEPCGPGPPQPGDSELPPRGNTNAARRPNSVQPGAEREKPGAGEGAGENWEPRVLPYHPAQPGQAAKKAVRTRYISTELGIRQRLLVAVLTSQTTLPTLGVAVNRTLGHRLERVVFLTGARGRRAPPGMAVVTLGEERPIGHLHLALRHLLEQHGDDFDWFFLVPDTTYTEAHGLARLTGHLSLASAAHLYLGRPQDFIGGEPTPGRYCHGGFGVLLSRMLLQQLRPHLEGCRNDIVSARPDEWLGRCILDATGVGCTGDHEGVHYSHLELSPGEPVQEGDPHFRSALTAHPVRDPVHMYQLHKAFARAELERTYQEIQELQWEIQNTSHLAVDGDRAAAWPVGIPAPSRPASRFEVLRWDYFTEQHAFSCADGSPRCPLRGADRADVADVLGTALEELNRRYHPALRLQKQQLVNGYRRFDPARGMEYTLDLQLEALTPQGGRRPLTRRVQLLRPLSRVEILPVPYVTEASRLTVLLPLAAAERDLAPGFLEAFATAALEPGDAAAALTLLLLYEPRQAQRVAHADVFAPVKAHVAELERRFPGARVPWLSVQTAAPSPLRLMDLLSKKHPLDTLFLLAGPDTVLTPDFLNRCRMHAISGWQAFFPMHFQAFHPAVAPPQGPGPPELGRDTGRFDRQAASEACFYNSDYVAARGRLAAASEQEEELLESLDVYELFLHFSSLHVLRAVEPALLQRYRAQTCSARLSEDLYHRCLQSVLEGLGSRTQLAMLLFEQEQGNST

SEQ ID NO: 5 - DNA, 1669 bp, Homo sapiens
  CDS (41)..(1024)
ctgcgagcgcctgccccatgcgccgccgcctctccgcacgatgttcccctcgcggaggaaagcggcgcagctgccctgggaggacggcaggtccgggttgctctccggcggcctccctcggaagtgttccgtcttccacctgttcgtggcctgcctctcgctgggcttcttctccctactctggctgcagctcagctgctctggggacgtggcccgggcagtcaggggacaagggcaggagacctcgggccctccccgcgcctgccccccagagccgccccctgagcactgggaagaagacgcatcctggggcccccaccgcctggcagtgctggtgcccttccgcgaacgcttcgaggagctcctggtcttcgtgccccacatgcgccgcttcctgagcaggaagaagatccggcaccacatctacgtgctcaaccaggtggaccacttcaggttcaaccgggcagcgctcatcaacgtgggcttcctggagagcagcaacagcacggactacattgccatgcacgacgttgacctgctccctctcaacgaggagctggactatggctttcctgaggctgggcccttccacgtggcctccccggagctccaccctctctaccactacaagacctatgtcggcggcatcctgctgctctccaagcagcactaccggctgtgcaatgggatgtccaaccgcttctggggctggggccgcgaggacgacgagttctaccggcgcattaagggagctgggctccagcttttccgcccctcgggaatcacaactgggtacaagacatttcgccacctgcacgacccagcctggcggaagagggaccagaagcgcatcgcagctcaaaaacaggagcagttcaaggtggacagggagggaggcctgaacactgtgaagtaccatgtggcttcccgcactgccctgtctgtgggcggggccccctgcactgtcctcaacatcatgttggactgtgacaagaccgccacaccctggtgcacattcagctgagctggatggacagtgaggaagcctgtacctacaggccatattgctcaggctcaggacaaggcctcaggtcgtgggcccagctctgacaggatgtggagtggccaggaccaagacagcaagctacgcaattgcagccacccggccgccaaggcaggcttgggctgggccaggacacgtggggtgcctgggacgctgcttgccatgcacagtgatcagagagaggctggggtgtgtcctgtccgggaccccccctgccttcctgctcaccctactctgacctccttcacgtgcccaggcctgtgggtagtggggagggctgaacaggacaacctctcatcacccccacttttgttccttcctgctgggctgcctcgtgcagagacacagtgtaggggccatgcagctggcgtaggtggcagttgggcctggtgagggttaggacttcagaaaccagagcacaagccccacagagggggaacagccagcaccgctctagctggttgttgccatgccggaatgtgggcctagtgttgccagatcttctgatttttcgaaagaaactagaatgctggattcttaagtgatatcttctgattttttaaatgatagcacctaaatgaaactttcaaaaagtaaaaaaaaaaa

SEQ ID NO: 6 - protein, 327 aa, Homo sapiens
MFPSRRKAAQLPWEDGRSGLLSGGLPRKCSVFHLFVACLSLGFFSLLWLQLSCSGDVARAVRGQGQETSGPPRACPPEPPPEHWEEDASWGPHRLAVLVPFRERFEELLVFVPHMRRFLSRKKIRHHIYVLNQVDHFRFNRAALINVGFLESSNSTDYIAMHDVDLLPLNEELDYGFPEAGPFHVASPELHPLYHYKTYVGGILLLSKQHYRLCNGMSNRFWGWGREDDEFYRRIKGAGLQLFRPSGITTGYKTFRHLHDPAWRKRDQKRIAAQKQEQFKVDREGGLNTVKYHVASRTALSVGGAPCTVLNIMLDCDKTATPWCTFS

SEQ ID NO: 7 - DNA, 26 bp, Artificial Sequence
  Description of Artificial Sequence: 5′ primer for PCR (K3)
cccaagcttgccgagggggagcccga

SEQ ID NO: 8 - DNA, 29 bp, Artificial Sequence
  Description of Artificial Sequence: 3′ primer for PCR (K3)
gctctagactgtcaggagagagttcgatt

SEQ ID NO: 9 - DNA, 25 bp, Artificial Sequence
  Description of Artificial Sequence: 5′ primer for PCR (K3)
atggctgtgcgctctcgccgcccgt

SEQ ID NO: 10 - DNA, 25 bp, Artificial Sequence
  Description of Artificial Sequence: 3′ primer for PCR (K3)
cgtccccgctgccgttgtggctact

SEQ ID NO: 11 - DNA, 25 bp, Artificial Sequence
  Description of Artificial Sequence: 5′ primer for PCR (K3)
agtagccacaacggcagcggggacg

SEQ ID NO: 12 - DNA, 24 bp, Artificial Sequence
  Description of Artificial Sequence: 3′ primer for PCR (K3)
tcaggagagagttcgattgtacct

SEQ ID NO: 13 - DNA, 28 bp, Artificial Sequence
  Description of Artificial Sequence: 5′ primer for PCR (K11)
ggaattccggccaggccgccaaaaaggc

SEQ ID NO: 14 - DNA, 30 bp, Artificial Sequence
  Description of Artificial Sequence: 3′ primer for PCR (K11)
cgggatcctcaggtgctgttgccctgctcc

SEQ ID NO: 15 - protein, 16 aa, Artificial Sequence
  Description of Artificial Sequence: synthesised substance for assay
VLPQEEEGSGGGQLVT

SEQ ID NO: 16 - DNA, 20 bp, Artificial Sequence
  Description of Artificial Sequence: 5′ primer for PCR and sequencing (K3 exon 1)
cgacagcccagcgagcgtcc

SEQ ID NO: 17 - DNA, 22 bp, Artificial Sequence
  Description of Artificial Sequence: 3′ primer for PCR and sequencing (K3 exon 1)
ggagactggcaggctggaaagc

SEQ ID NO: 18 - DNA, 20 bp, Artificial Sequence
  Description of Artificial Sequence: Primer for sequencing (K3 exon 1)
agggggagcccgaggaggag

SEQ ID NO: 19 - DNA, 20 bp, Artificial Sequence
  Description of Artificial Sequence: Primer for sequencing (K3 exon 1)
ctcctcctcgggctccccct

SEQ ID NO: 20 - DNA, 27 bp, Artificial Sequence
  Description of Artificial Sequence: 5′ primer for PCR and sequencing (K3 exon 2)
gagacatagtaattgttgcctttcttt

SEQ ID NO: 21 - DNA, 25 bp, Artificial Sequence
  Description of Artificial Sequence: 3′ primer for PCR and sequencing (K3 exon 2)
gtgaacattttcatcacagctccat

SEQ ID NO: 22 - DNA, 26 bp, Artificial Sequence
  Description of Artificial Sequence: 5′ primer for PCR and sequencing (K3 exon 3)
tagatgctttagtttatcgctggttt

SEQ ID NO: 23 - DNA, 26 bp, Artificial Sequence
  Description of Artificial Sequence: 3′ primer for PCR and sequencing (K3 exon 3)
ttaaaaaaggcaaaatgtgttgcctg

SEQ ID NO: 24 - DNA, 24 bp, Artificial Sequence
  Description of Artificial Sequence: Primer for sequencing (K3 exon 3)
tctatactcagcagctgagaacca

SEQ ID NO: 25 - DNA, 24 bp, Artificial Sequence
  Description of Artificial Sequence: Primer for sequencing (K3 exon 3)
tggttctcagctgctgagtataga

SEQ ID NO: 26 - DNA, 24 bp, Artificial Sequence
  Description of Artificial Sequence: Primer for sequencing (K3 exon 3)
gaaatgggagggcacaatgaaaag

SEQ ID NO: 27 - DNA, 24 bp, Artificial Sequence
  Description of Artificial Sequence: Primer for sequencing (K3 exon 3)
cttttcattgtgccctcccatttc

SEQ ID NO: 28 - DNA, 24 bp, Artificial Sequence
  Description of Artificial Sequence: Primer for sequencing (K3 exon 3)
tagccagtatgacccaaaggtaac

SEQ ID NO: 29 - DNA, 24 bp, Artificial Sequence
  Description of Artificial Sequence: Primer for sequencing (K3 exon 3)
gttacctttgggtcatactggcta

SEQ ID NO: 30 - DNA, 31 bp, Artificial Sequence
  Description of Artificial Sequence: Primer for sequencing (K3 exon 3)
aggccattcagaagccaagaagtaggagtgg

SEQ ID NO: 31 - DNA, 31 bp, Artificial Sequence
  Description of Artificial Sequence: Primer for sequencing (K3 exon 3)
ccactcctacttcttggcttctgaatggcct

SEQ ID NO: 32 - DNA, 21 bp, Artificial Sequence
  Description of Artificial Sequence: 5′ primer for PCR and sequencing (K11 exon 1)
tcggagactcctctggctgct

SEQ ID NO: 33 - DNA, 21 bp, Artificial Sequence
  Descriprion of Artificial Sequence: 3′ primer for PCR and sequencing (K11 exon 1)
tagagcgggcgcagccgatca

SEQ ID NO: 34 - DNA, 25 bp, Artificial Sequence
  Description of Artificial Sequence: 5′ primer for PCR and sequencing (K11 exon 2)
tttgataagcttgtgccatctcctc

SEQ ID NO: 35 - DNA, 25 bp, Artificial Sequence
  Description of Artificial Sequence: 3′ primer for PCR and sequencing (K11 exon 2)
aggtatcagtgggatagcttatcat

SEQ ID NO: 36 - DNA, 24 bp, Artificial Sequence
  Description of Artificial Sequence: 5′ primer for PCR and sequencing (K11 exon 3)
agctcatcacagatcccttccctt

SEQ ID NO: 37 - DNA, 22 bp, Artificial Sequence
  Description of Artificial Sequence: 3′ primer for PCR and sequencing (K11 exon 1)
actctgccacccccagacctag

SEQ ID NO: 38 - DNA, 24 bp, Artificial Sequence
  Description of Artificial Sequence: 5′ primer for PCR and sequencing (K11 exon 4)
ttgctgatggcctgtttctctgat

SEQ ID NO: 39 - DNA, 21 bp, Artificial Sequence
  Description of Artificial Sequence: 3′ primer for PCR and sequencing (K11 exon 4)
gtgtggccatgccacggccca

SEQ ID NO: 40 - DNA, 23 bp, Artificial Sequence
  Description of Artificial Sequence: Primer for sequencing (K11 exon 4)
tatgtcactgaggcctcacgtct

SEQ ID NO: 41 - DNA, 23 bp, Artificial Sequence
  Description of Artificial Sequence: Primer for sequencing (K11 exon 4)
agacgtgaggcctcagtgacata

SEQ ID NO: 42 - DNA, 24 bp, Artificial Sequence
  Description of Artificial Sequence: primer for sequencing (K11 exon 4)
atgcatttccaagccttccaccca

SEQ ID NO: 43 - DNA, 24 bp, Artificial Sequence
  Description of Artificial Sequence: Primer for sequencing (K11 exon 4)
tgggtggaaggcttggaaatgcat

SEQ ID NO: 44 - DNA, 20 bp, Artificial Sequence
  Description of Artificial Sequence: 5′ primer for PCR and sequencing (beta4Gal-T7 exon 1)
tgcgagcgcctgccccatgc

SEQ ID NO: 45 - DNA, 22 bp, Artificial Sequence
  Description of Artificial Sequence: 3′ primer for PCR and sequencing (beta4Gal-T7 exon 1)
gatggcctcgggttcccagatt

SEQ ID NO: 46 - DNA, 21 bp, Artificial Sequence
  Description of Artificial Sequence: 5′ primer for PCR and sequencing (beta4Gal-T7 exon 2)
tcctgaccctgtcccgcgctt

SEQ ID NO: 47 - DNA, 20 bp, Artificial Sequence
  Description of Artificial Sequence: 3′ primer for PCR and sequencing (beta4Gal-T7 exon 2)
aggggtgccgaggggagagg

SEQ ID NO: 48 - DNA, 21 bp, Artificial Sequence
  Description of Artificial Sequence: 5′ primer for PCR and sequencing (beta4Gal-T7 exon 3)
ctgcccagccttgcccaccct

SEQ ID NO: 49 - DNA, 22 bp, Artificial Sequence
  Description of Artificial Sequence: 3′ primer for PCR and sequencing (beta4Gal-T7 exon 3)
gctctgagcagagcaggctgtc

SEQ ID NO: 50 - DNA, 22 bp, Artificial Sequence
  Description of Artificial Sequence: 5′ primer for PCR and sequencing (beta4Gal-T7 exon 4)
agatgggccgagtgacgctgct

SEQ ID NO: 51 - DNA, 21 bp, Artificial Sequence
  Description of Artificial Sequence: 3′ primer for PCR and sequencing (beta4Gal-T7 exon 4)
ctcagggcagccaccgcagct

SEQ ID NO: 52 - DNA, 22 bp, Artificial Sequence
  Description of Artificial Sequence: 5′ primer for PCR and sequencing (beta4Gal-T7 exon 5)
aagggcagcctgaccccgactt

SEQ ID NO: 53 - DNA, 23 bp, Artificial Sequence
  Description of Artificial Sequence: 3′ primer for PCR and sequencing (beta4Gal-T7 exon 5)
atgaccacctatccgtccccaat

SEQ ID NO: 54 - DNA, 22 bp, Artificial Sequence
  Description of Artificial Sequence: 5′ primer for PCR and sequencing (beta4Gal-T7 exon 6)
cagccctgagtccgtgctcttt

SEQ ID NO: 55 - DNA, 23 bp, Artificial Sequence
  Description of Artificial Sequence: 3′ primer for PCR and sequencing (beta4Gal-T7 exon 6)
tggcctgtaggtacaggcttcct

SEQ ID NO: 56 - DNA, 23 bp, Artificial Sequence
  Description of Artificial Sequence: 5′ primer for RT-P
cccagaaaaagtccttcatgatg

SEQ ID NO: 57 - DNA, 30 bp, Artificial Sequence
  Description of Artificial Sequence: 3′ primer for RT-P
aactcttctaatttgtcacctttgatgtag

SEQ ID NO: 58 - DNA, 17 bp, Artificial Sequence
  Description of Artificial Sequence: Probe for RT-PC
atgagtggttcatgcgc

SEQ ID NO: 59 - DNA, 20 bp, Artificial Sequence
  Description of Artificial Sequence: 5′ primer for RT-PC
gctgaactggaacgcacgta

SEQ ID NO: 60 - DNA, 19 bp, Artificial Sequence
  Description of Artificial Sequence: 3′ primer for RT-PC
cgggatggtgctggaatac

SEQ ID NO: 61 - DNA, 20 bp, Artificial Sequence
  Description of Artificial Sequence: Probe for RT-PCR
agatccaggagttacagtgg

SEQ ID NO: 62 - DNA, 18 bp, Artificial Sequence
  Description of Artificial Sequence: 5′ primer for RT-PCR  (beta4Gal-T7)
cggcgcattaagggagct

SEQ ID NO: 63 - DNA, 21 bp, Artificial Sequence
  Description of Artificial Sequence: 3′ primer for RT-PCR  (beta4Gal-T7)
tacccagttgtgattcccgag

SEQ ID NO: 64 - DNA, 14 bp, Artificial Sequence
  Description of Artificial Sequence: Probe for RT-PCR (beta4Gal-T7)
ctccagcttttccg

SEQ ID NO: 65 - DNA, 2652 bp, mouse
  CDS (1)..(2652)
atggccgtgcgctcccgccgcccatgggtgagcgtggcattggggttggtcctgggcttcaccgccgcgtcctggctcatcgccccccgggtggccgagctgagcgagaagaggcgacgcggctccagtctttgctcctactacggccgctcggctaccgggccccgcgcggacgcgcagcagctgctcccccaaccccagtcccggccgcggctagagcagtcgccgccccctgccagccacgagctccccggtcctcagcagccggaggcggcgcccggaggtcccagttttcggagcagcccctggcagcagccggctctgttgccgcagaggaggcgaggacacacgcccgaaggtgcgacggcgcttcccggcgctccggctgccaaaggggaaccagaggaggaggatgggggcgcggctgaccctcggaagggtggccggccggggagcagccacaacggcagcggggacgggggtgccgctgtcccgacctccggacccggggacttcctgtacgtgggtgtgatgaccgcacagaagtacctgggcagtcgcgcgctggccgcgcagcggacctgggcgcgcttcatccctggccgcgtggagttcttttccagtcagcaatctcccagtgctgcgcttggccagcccccgccacctttgcctgtcatcgcgctgccaggggtcgacgattcctaccctccccagaaaaagtccttcatgatgatcaagtacatgcacgaccactatctggacaagtatgagtggttcatgcgcgccgacgacgatgtctacatcaaaggtgataagttagaagaatttctaagatccctaaatagcagcaagcctctctacctgggacagacgggcctgggcaatactgaagaacttggaaagctggggctggagcccggggagaacttctgcatgggaggacctggcatgatcttcagcagagaggttctcaggcggatggtgcctcatatcggcgaatgcctccgagagatgtacaccacacacgaagacgtagaagtaggaaggtgtgttcgccgtttcggcgggacgcagtgtgtctggtcttatgagatgcagcagctgttccatgaaaactacgaacacaatcgcaagggttacatccaagacctccacaacagcaaaatccacgcagccatcacgctccatccgaacaaaaggcccgcgtaccagtacagacttcataactacatgctcagccgcaagatctccgagctccgctaccgcaccatccagctccaccgggagagcgctctcatgagcaagctcagcaacagtgaagtgagcaaagaggaccaacagctgggaaggacgccgtccttcaaccacttccagcctcgggagagaaatgaagtcatggagtgggagttcctgacggggaagctgctttactcagctgcagagaaccagcctcctcgacagagcatcaacagcatcctaaggtcagctctggatgacactgtcctgcaggtgatggagatgatcaacgagaatgccaagagtaggggccgactcatcgacttcaaggaaattcagtatggctaccgcagggttgatcccatgcacggggttgagtacatattggacctgctactcctgtacaaaaggcacaaaggaaggaaactgactgtgcctgtgaggcgccatgcctatcttcagcagccatttagcaagcctttcttcagagaggtggaagaactcgacgtcaaccgtctggtggagagtatcaacagcggtacacagtcattctccgttatatccaattctctaaaaattctctcttctcttcaagaggccaaagacataggagggcacaatgaaaagaaagtacacattctcgttccactcgttggaaggtacgacattttcttgagattcatggaaaattttgaaagtacgtgtcttatcccaaagcaaaatgtcaagcttgtcatcatccttttcagcagggatgctggccaagagtccatcaagcacattgagctgatacaagaatatcagagcaggtatcccagtgcagaaatgatgctcattcccatgaagggagagttttccagaggtcttggtcttgaaatggcttcttcccagtttgacaatgacacattgctgctattttgtgatgttgacttgattttcagaggagacttcctccaacgctgtcgagacaatacagttcagggacaacaggtatattaccccatcatctttagccagtatgacccaaaggtcacccatatgagaaatcctcccacagagggtgactttgtattctcaaaggaaactgggttttggagagactatggctacggaatcacatgcatttacaaaagcgatctactgggtgcaggtggatttgatacctcaatacaaggctggggactggaagatgtagatctctataataaagtcatcctatctggcttacggcccttcagaagtcaagaagtgggagtggtgcatattttccatcctgttcattgtgatcctaacttggaccctaagcagtataagatgtgcttaggatccaaagcaagtacttttgcctcaaccatgcaactggctgaactctggttagaaaaacatttgggtgtcagggataatcgaactctctcc

SEQ ID NO: 66 - protein, 884 aa, mouse
MAVRSRRPWVSVALGLVLGFTAASWLIAPRVAELSEKRRRGSSLCSYYGRSATGPRADAQQLLPQPQSRPRLEQSPPPASHELPGPQQPEAAPGGPSFRSSPWQQPALLPQRRRGHTPEGATALPGAPAAKGEPEEEDGGAADPRKGGRPGSSHNGSGDGGAAVPTSGPGDFLYVGVMTAQKYLGSRALAAQRTWARFIPGRVEFFSSQQSPSAALGQPPPPLPVIALPGVDDSYPPQKKSFMMIKYMHDHYLDKYEWFMRADDDVYIKGDKLEEFLRSLNSSKPLYLGQTGLGNTEELGKLGLEPGENFCMGGPGMIFSREVLRRMVPHIGECLREMYTTHEDVEVGRCVRRFGGTQCVWSYEMQQLFHENYEHNRKGYIQDLHNSKIHAAITLHPNKRPAYQYRLHNYMLSRKISELRYRTIQLHRESALMSKLSNSEVSKEDQQLGRTPSFNHFQPRERNEVMEWEFLTGKLLYSAAENQPPRQSINSILRSALDDTVLQVMEMINENAKSRGRLIDFKEIQYGYRRVDPMHGVEYILDLLLLYKRHKGRKLTVPVRRHAYLQQPFSKPFFREVEELDVNRLVESINSGTQSFSVISNSLKILSSLQEAKDIGGHNEKKVHILVPLVGRYDIFLRFMENFESTCLIPKQNVKLVIILFSRDAGQESIKHIELIQEYQSRYPSAEMMLIPMKGEFSRGLGLEMASSQFDNDTLLLFCDVDLIFRGDFLQRCRDNTVQGQQVYYPIIFSQYDPKVTHMRNPPTEGDFVFSKETGFWRDYGYGITCIYKSDLLGAGGFDTSIQGWGLEDVDLYNKVILSGLRPFRSQEVGVVHIFHPVHCDPNLDPKQYKMCLGSKASTFASTMQLAELWLEKHLGVRDNRTLS

SEQ ID NO: 67 - DNA, 2490 bp, mouse
  CDS (1)..(2325)
atgcgggcgtcgctgctgctgtccgtgctgcggcccgcggggcccgtggccgtgggcatctctctgggcttcaccctgagcctgctcagcgtcacctgggtggaggagccttgcggacccgggccgccccaacccggagactctgagctgccgccgcgcggcaacaccaacgcggcgcgccggcccaactcggtgcagcccggatccgagcgcgagaggcccggggccggcgcaggcaccggtgagagctgggagcctcgtgtcttgccctaccatcccgcgcagccaggccaggccaccaagaaggccgtcagaactcggtatatcagcacggagctgggcatcaggcagaagcttctggtggcagtgctgacctcacaagccacgttgcctacactgggtgtggctgtaaaccgaactctgggacaccgactggagcatgtagtgttcctgaccggtgcgaggggccgccggacaccttcaggcatggcggtggtggcactgggcgaagagaggcccatcggacacctgcacctggcgctgcgccacctgctggagcaacacggcgatgactttgactggtttttcctagtgcctgatgccacctatactgaagcgcatggactggaccgcctagctggccacctcagccttgcttcagcaacccatctctatcttggccggccgcaggacttcatcggtggagatactaccccaggccgctactgccacgggggctttggagtcttgctctctcgcacactgctacagcaactgcgcccccacctggaaagctgccgcaacgacatcgtcagtgctcgcccggatgagtggttgggccgctgcatccttgatgccacaggcgtgggctgtactggtgaccacgagggaatgcactacaactacctggaactgagccccggggagcctgtacaggagggggaccctcgtttccgcagcgccttgacagcccatcccgtgcgtgaccctgtgcacatgtaccagctgcacaaagcttttgcccgcgctgagctggaccgcacgtaccaggagattcaagaattgcagtgggagatccagaataccagccgactggctgctgatggggagagagcctctgcctggccagtgggcatcccagcaccgtctcgccctgcctcacgctttgaggttctgcgctgggactacttcacagaacaatacgcgttctcctgcgccgatggctctccccgctgcccgttgcgtggggccgaccaggctgatgtggctgacgtcctggggacagccttagaggagctcaaccgccgttaccagccagcgctgcagctccagaagcaacagctggtgaacggctaccggcgttttgatccagcccgaggcatggagtacacactagacctgcagctggaagcgctgacaccccagggtggccgctggcccctcacccgcagggtgcagctccttcggcccttgagccgagtggagatcttgcctgtaccctatgtcaccgaggcttctcggctcactgtgctactgccgctggctgcagcggaacgagacctggcttctggcttcttagaagcctttgccactgcagccctggaacctggtgatgcagcagccttgaccctgctgctgctgtatgagccacgccaggcccagcgggcagcccactcagacgtcttcgcacctgtcaaggcccacgtggcagagctagagcggcgtttccctggtgcccgggtgccctggctcagtgtgcagacagcagcgccctctccactgcgtctcatggatctgctgtccaagaagcacccactagacactctgttcctgctggccgggccagacacggtactcacacctgatttcctgaaccgctgccgcatgcatgccatctctggctggcaggccttcttccccatgcacttccaggccttccaccctgctgtggctcctcctcagggccctgggccaccagagctgggccgtgacaccggtcactttgatcgccaggctgccagtgaggcatgcttctacaactccgactatgtggcggcccgtggccggctggtggcggcctcggagcaggaggaggagctgctggagagcctggatgtgtacgagttgtttctgcgcttctccaacttgcacgtgctgagagcagtagagccagccttgctgcagcgctaccgggcccagccgtgcagtgcacggctcagtgaagacctttaccaccgctgtcgccagagcgtacttgagggccttggctcccgcacccagcttgccatgctgctctttgagcaggaacaggggaacagcacctaagcccctgcacctgtccctgctcttccccaggaacctggagccacgtgccagcctcgctggacagggctggctgtagcctcagtccctagggcagcccactggtcccttgtctcttgctttgttggacccatgggctcaggacaagccctgagacagatgccctag

SEQ ID NO: 68 - protein, 774 aa, mouse
MRASLLLSVLRPAGPVAVGISLGFTLSLLSVTWVEEPCGPGPPQPGDSELPPRGNTNAARRPNSVQPGSERERPGAGAGTGESWEPRVLPYHPAQPGQATKKAVRTRYISTELGIRQKLLVAVLTSQATLPTLGVAVNRTLGHRLEHVVFLTGARGRRTPSGMAVVALGEERPIGHLHLALRHLLEQHGDDFDWFFLVPDATYTEAHGLDRLAGHLSLASATHLYLGRPQDFIGGDTTPGRYCHGGFGVLLSRTLLQQLRPHLESCRNDIVSARPDEWLGRCILDATGVGCTGDHEGMHYNYLELSPGEPVQEGDPRFRSALTAHPVRDPVHMYQLHKAFARAELDRTYQEIQELQWEIQNTSRLAADGERASAWPVGIPAPSRPASRFEVLRWDYFTEQYAFSCADGSPRCPLRGADQADVADVLGTALEELNRRYQPALQLQKQQLVNGYRRFDPARGMEYTLDLQLEALTPQGGRWPLTRRVQLLRPLSRVEILPVPYVTEASRLTVLLPLAAAERDLASGFLEAFATAALEPGDAAALTLLLLYEPRQAQRAAHSDVFAPVKAHVAELERRFPGARVPWLSVQTAAPSPLRLMDLLSKKHPLDTLFLLAGPDTVLTPDFLNRCRMHAISGWQAFFPMHFQAFHPAVAPPQGPGPPELGRDTGHFDRQAASEACFYNSDYVAARGRLVAASEQEEELLESLDVYELFLRFSNLHVLRAVEPALLQRYRAQPCSARLSEDLYHRCRQSVLEGLGSRTQLAMLLFEQEQGNST

SEQ ID NO: 69 - DNA, 984 bp, mouse
  CDS (1)..(984)
atgttgccctcccggaggaaagcggcgcagctgccctgggaggatggcagggccaggttgcttcctggaggcctccgccggaaatgctccatcttccacctcttcattgcctttctcctgttggtcttcttctccctgctctggctgcagctcagctgttctggagatatggcccaggtgaccaggggacaagggcaagagacctcgggtccaccccgggcttgccctccagagccgccccctgagcactgggaagaagatgagtcctgggggccccaccgcttggcagtgctggtgccctttcgtgagcgctttgaggagctgctggtctttgtgccccacatgcaccgcttcctaagcaggaagaggatccagcaccacatctatgtgctcaaccaggtggaccatttcaggttcaatcgggcagcactcatcaatgtgggcttcctggagagcagcaacagcacagactacatcgccatgcacgatgtggacctgctccctctcaatgaggagctggactatggcttcccggaggctgggcccttccatgtggcctccccagagctccaccctctctaccactacaagacctatgtgggcggcattctgctgctgtccaaacagcactaccagctgtgcaacggaatgtccaaccgcttttggggctggggccgagaggatgatgaattctaccggcgcatcaaaggagctggcctccagcttttccgcccctcgggaatcacaactgggtaccagacatttcgccacttgcatgaccctgcctggcggaagagggaccaaaaacgcattgcggctcaaaaacaggaacagttcaaggtggaccgggagggaggcctgaacactgtgaagtaccgggtggattcccgcacggcactgtctataggaggggccccgtgcactgtcctcaatgtcatgctggactgcgataaaacagccaccccatggtgcatatttggctga

SEQ ID NO: 70 - protein, 327 aa, mouse
MLPSRRKAAQLPWEDGRARLLPGGLRRKCSIFHLFIAFLLLVFFSLLWLQLSCSGDMAQVTRGQGQETSGPPRACPPEPPPEHWEEDESWGPHRLAVLVPFRERFEELLVFVPHMHRFLSRKRIQHHIYVLNQVDHFRFNRAALINVGFLESSNSTDYIAMHDVDLLPLNEELDYGFPEAGPFHVASPELHPLYHYKTYVGGILLLSKQHYQLCNGMSNRFWGWGREDDEFYRRIKGAGLQLFRPSGITTGYQTFRHLHDPAWRKRDQKRIAAQKQEQFKVDREGGLNTVKYRVDSRTALSIGGAPCTVLNVMLDCDKTATPWCIFG

(19) United States
(12) Patent Application Publication
(10) Pub. No.: US 2006/0141463 A1
(43) Pub. Date: Jun. 29, 2006

(54) Method of detecting bone paget's disease

(76) Inventors: Hisashi Narimatsu, Tsukuba-shi (JP); Takashi Sato, Tsukuba-shi (JP); Masanori Gotoh, Tsukuba-shi (JP)

Correspondence Address: NIXON & VANDERHYE, PC, 901 NORTH GLEBE ROAD, 11TH FLOOR, ARLINGTON, VA, 22203, US

(21) Appl. No.: 10/534,002

(22) PCT Filed: Nov. 7, 2003
(86) PCT No.: PCT/JP03/14211

§ 371 Date: Feb. 17, 2006

(30) Foreign Application Priority Data
Nov. 7, 2002 (JP) 2002-323438

(51) Int. Cl. C12Q 1/68; A01K 67/027

(52) U.S. Cl. 435/6; 800/18

(57) ABSTRACT

A method for detecting Paget disease of bone and an animal showing the pathology of Paget disease of bone are provided. In particular, there are provided associating Paget disease of bone with the mutation a chondroitin/chondroitin synthase gene comprising the amino acid sequence depicted in any one of SEQ ID NO: 2, SEQ ID NO: 4, SEQ ID NO: 6, SEQ ID NO: 66, SEQ ID NO: 68 or SEQ ID NO: 70 or the amount of expression of said gene, detection of Paget disease of bone, and a knockout animal prepared by knocking out the chondroitin/chondroitin synthase gene.

### TECHNICAL FIELD

This invention relates to a method of detecting Paget disease of bone and, more specifically, to a method of detecting Paget disease of bone by associating Paget disease of bone with the mutation in a gene coding for an enzyme that takes part in the synthesis of chondroitin/chondroitin sulfate or the amount of expression of said gene.

### BACKGROUND ART

Paget disease of bone is a disease characterized by concomitant increase of osteoclasts and osteoblasts which leads to localized abnormal enhancement of bone resorption and the accompanying secondary bone formation, eventually causing bone deformation and thickening. It is held predominantly that the onset of Paget disease of bone is triggered by a viral infection and Reference 1 states that measles virus could be detected by reverse transcription-polymerase chain reaction method (RT-PCR method) using RNA extracted from peripheral blood- and bone marrow-derived mononuclear cells from patients. However, the reference does not show that measles virus is a direct cause of Paget disease of bone and the pathological etiology for Paget disease of bone is unknown. Patients with this disease are rare in Japan but a lot of patients are found in Europe and the United States of America and according to Reference 2, the incidence of the disease increases with age and is higher in men than in women.

Mutant gene loci that are responsible for Paget disease of bone are already known and described in Reference 3; however, more than one gene is present on those loci and it has not been known which gene should mutate to induce Paget disease of bone. Therefore, in order to diagnose Paget disease of bone by a convenient method, it has been necessary to identify which gene should mutate for the disease to manifest itself.

### DISCLOSURE OF THE INVENTION

There has been a growing expectation for scientists to identify the causative gene responsible for the pathology of Paget disease of bone and to develop a convenient method for detecting Paget disease of bone.

### BRIEF DESCRIPTION OF THE DRAWINGS

FIG. 1 shows a chromatographic chart for the synthesis of odd-numbered saccharides by the GalNAc transfer activity of K3; open circles show the activity for transferring GalNAc to chondroitin sulfate; solid circles show the activity for transferring GalNAc to chondroitin; open triangles show the activity for transferring GalNAc to chondroitin sulfate decasaccharide; and solid triangles show the activity for transferring GalNAc to chondroitin decasaccharide.

FIG. 2 shows a chromatographic chart for the undecasaccharide prepared by the GalNAc transfer activity of K3 and the product of its digestion with chondroitinase ACII; open circles refer to the undecasaccharide not digested with chondroitinase ACII; and solid circles refer to the product of digestion with chondroitinase ACII.

FIG. 3 shows a chromatographic chart for the synthesis of even-numbered saccharides by the GlcUA transfer activity of K3; open circles show the activity for transferring GlcUA to chondroitin sulfate; solid circles show the activity for transferring GlcUA to chondroitin; open triangles show the activity for transferring GlcUA to chondroitin sulfate undecasaccharide; and solid triangles show the activity for transferring GlcUA to chondroitin undecasaccharide.

FIG. 4 shows a chromatographic chart for the dodecasaccharide prepared by the GlcUA transfer activity of K3 and the product of its digestion with chondroitinase ACII; open circles refer to the dodecasaccharide not digested with chondroitinase ACII; and solid circles refer to the product of digestion with chondroitinase ACII.

FIG. 5 shows a chromatographic chart for the synthesis of odd-numbered saccharides by the GalNAc transfer activity of K11; open circles show the activity for transferring GalNAc to chondroitin sulfate; solid circles show the activity for transferring GalNAc to chondroitin; open triangles show the activity for transferring GalNAc to chondroitin sulfate decasaccharide; and solid triangles show the activity for transferring GalNAc to chondroitin decasaccharide.

FIG. 6 shows a chromatographic chart for the undecasaccharide prepared by the GalNAc transfer activity of K11 and the product of its digestion with chondroitinase ACII; open circles refer to the undecasaccharide not digested with chondroitinase ACII; and solid circles refer to the product of digestion with chondroitinase ACII.

FIG. 7 shows a chromatographic chart for the synthesis of even-numbered saccharides by the GlcUA transfer activity of K11; open circles show the activity for transferring GlcUA to chondroitin sulfate; solid circles show the activity for transferring GlcUA to chondroitin; open triangles show the activity for transferring GlcUA to chondroitin sulfate undecasaccharide; and solid triangles show the activity for transferring GlcUA to chondroitin undecasaccharide.

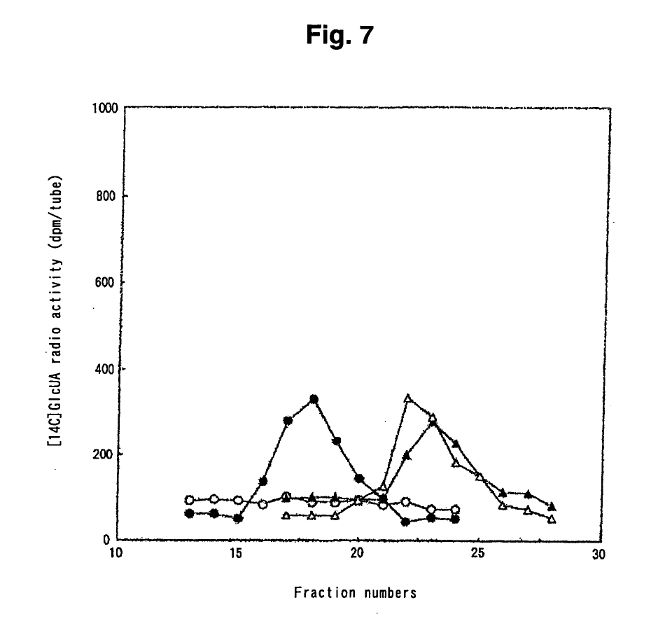

FIG. 8 shows a chromatographic chart for the dodecasaccharide prepared by the GlcUA transfer activity of K11 and the product of its digestion with chondroitinase ACII; open circles refer to the dodecasaccharide not digested with chondroitinase ACII; and solid circles refer to the product of digestion with chondroitinase ACII.

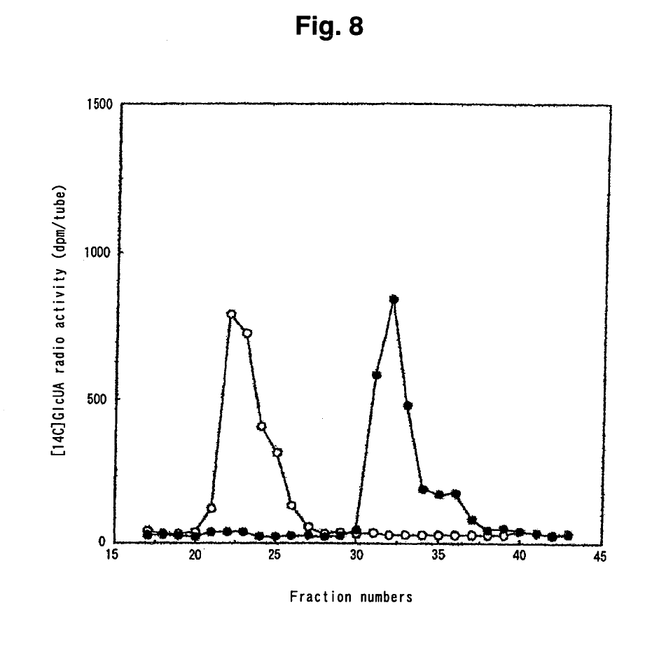

### DETAILED DESCRIPTION OF THE INVENTION

The present inventors made intensive studies with a view to solving the above-described problem and found that each of the gene mutations leading to Paget disease of bone occurred in the locus of DNA that would take part in the synthesis of chondroitin/chondroitin sulfate; the inventors applied this finding to the detection of Paget disease of bone and eventually completed the present invention.

The present invention is summarized as a method of detecting Paget disease of bone by associating the disease with the mutation in a gene coding a chondroitin synthase or the amount of expression of said gene.

### MODES FOR CARRYING OUT THE INVENTION

On the pages that follow, the present invention is described in detail by reference to various modes for carrying out it.

The detection method of the present invention is one for detecting Paget disease of bone by associating Paget disease of bone with the mutation of a gene coding a chondroitin synthase or the amount of expression of said gene.

The “chondroitin synthase” that can be used in the detection method of the present invention is not limited in any particular way as long as it is an enzyme that takes part in the synthesis of chondroitin or chondroitin sulfate. To be more specific, chondroitin and chondroitin sulfate are usually synthesized in vivo as proteoglycans, so not only enzymes that take part in extending the building blocks of chondroitin/chondroitin sulfate but also other enzymes such as those that synthesize the linkage between the protein of proteoglycan and glycosaminoglycan are encompassed. If these enzymes mutate or if they are not expressed, there will be no synthesis of chondroitin/chondroitin sulfate.

The “chondroitin synthase” that can be used in such detection method of the present invention is preferably a glycosyltransferase having activity for transferring a D-glucuronic acid residue or an N-acetyl-D-galactosamine residue to the saccharide residue at the non-reducing terminal of chondroitin/chondroitin sulfate (which may hereunder be also designated “glycosyltransferase 1” for convenience sake) or a glycosyltransferase by means of which a xylose residue linked to an amino acid residue has D-galactose linked thereto by a β1,4-glycoside linkage (which may hereunder be also designated “glycosyltransferase 2” for convenience sake).

The “glycosyltransferase 1” mentioned above is not limited in any particular way as long as it has activity for transferring a D-glucuronic acid (hereunder also referred to simply as GlcUA) residue or an N-acetyl-D-galactosamine (hereunder also referred to simply as N-acetylgalactosamine or GalNAc) residue to the saccharide residue at the non-reducing terminal of chondroitin/chondroitin sulfate. Such glycosyltransferase 1 takes part in extending chondroitin/chondroitin sulfate and if it mutates or is suppressed in expression, normal synthesis of chondroitin/chondroitin sulfate is believed to be prevented, leading to Paget disease of bone which can cause abnormalities in bone formation.

“Chondroitin” as it appears in the above definition of “glycosyltransferase 1” is a kind of glycosaminoglycan having the building blocks in which disaccharides each composed of a uronic acid (hereunder also referred to simply as “UA”) residue and a GalNAc residue are linked by a 1,3 glycoside linkage are repeatedly linked by a β1,4 glycoside linkage and which are represented by -[4UA1-3GalNAcβ1-]ₙ (n is an integer of 2 or more). Chondroitin has no sulfuric acid group linked to these building blocks but chondroitin sulfate has the carbon atom sulfated at 2-, 4- or 6-position of the GalNAc residue or at 2-position of the uronic acid residue.

The “saccharide residue at the non-reducing terminal of chondroitin/chondroitin sulfate” is any one of the GalNAc residue and the UA residue that make up the building blocks of chondroitin/chondroitin sulfate, and “glycosyltransferase 1” is an enzyme that transfers UA when the saccharide residue under consideration is the GalNAc residue and transfers GalNAc when it is the UA residue but which at least has the activity for transferring either UA or GalNAc.

Specific examples of such “glycosyltransferase 1” include a chondroitin synthase containing a protein comprising the amino acid sequence of amino acid Nos. 1-882 depicted as SEQ ID NO:2.

Said enzyme has the activity for transferring the GalNAc residue from a GalNAc donor substrate (e.g. UDP-GalNAc) to a GalNAc acceptor containing a structure represented by formula 1 shown below to synthesize a compound containing a structure represented by formula 2 shown below, as well as the activity for transferring the GlcUA residue from a GlcUA donor substrate (e.g. UDP-GlcUA) to a GlcUA acceptor containing a structure represented by formula 3 shown below to synthesize a compound containing a structure represented by formula 4 shown below (see Reference Example 2 in this specification).
GlcUAβ1-(3GalNAcβ1-4GlcUAβ1)ₖ-(3GalNAc)ₗ (1)
GalNAcβ1-4GlcUAβ1-(3GalNAcβ1-4GlcUAβ1)ₖ-(3GalNAc)ₗ (2)
GalNAcβ1-(4GlcUAβ1-3GalNAcβ1)ₘ-(4GlcUA)ₙ (3)
GlcUAβ1-3GalNAcβ1-(4GlcUAβ1-3GalNAcβ1)ₘ-(4GlcUA)ₙ (4)

In formulas 1-4, “-” represents a glycoside linkage and the numerals designate the carbon positions in the saccharide ring at which said glycoside linkage is located; “α” and “β” refer to anomers having said glycoside linkage at 1-position of the saccharide ring, with “α” having a trans-configuration to CH₂OH or COOH at 5-position and “β” having a cis-configuration; k and m are each an integer of 1 or more; l and n are each 1 or 0.

The gene for the enzyme under consideration which comprised the nucleotide sequence of SEQ ID NO:1 was subjected to a locus search using OMIM (Online Mendelian Inheritance in Man) and it was found to exist at 5q31.1 (see Reference Example 1).

Another example of “glycosyltransferase 1” is a chondroitin synthase containing a protein comprising the amino acid sequence of amino acid Nos. 1-755 depicted in SEQ ID NO:4″.

Said enzyme has the activity for transferring the GalNAc residue from a GalNAc donor (e.g. UDP-GalNAc) to a GalNAc acceptor containing a structure represented by formula 1 shown above to synthesize a compound containing a structure represented by formula 2 shown above, as well as the activity for transferring the GlcUA residue from a GlcUA donor (e.g. UDP-GlcUA) to a GlcUA acceptor containing a structure represented by formula 3 shown above to synthesize a compound containing a structure represented by formula 4 shown above (see Reference Example 4 in this specification).

The gene for the enzyme under consideration which comprised the nucleotide sequence of SEQ ID NO:3 was subjected to a locus search using OMIM and it was found to exist at 2q36.3 (see Reference Example 3 in this specification).

Speaking of the “activity for transferring a GlcUA residue or a GalNAc residue to the saccharide residue at the non-reducing terminal of chondroitin/chondroitin sulfate”, a GlcUA donor substrate or a GalNAc donor substrate that are labeled, for example, with radioactivity and a non-radiolabeled acceptor substrate for chondroitin/chondroitin sulfate, which may be reduced in molecular weight to make lower molecular weight chondroitin/lower molecular weight chondroitin sulfate (sugar chains not longer than eicosasaccharide), may be used to perform enzymatic reaction and the activity under consideration can be easily detected by subjecting the reaction product to gel filtration and analysis with an autoradiograph or scintillation counter. The above-mentioned GlcUA donor substrate is preferably a sugar nucleotide having GlcUA and may be exemplified by adenosine diphosphate-D-glucuronic acid (ADP-GlcUA), uridine diphosphate-D-glucuronic acid (UDP-GlcUA), guanosine diphosphate-D-glucuronic acid (GDP-GlcUA), cytidine diphosphate-D-glucuronic acid (CDP-GlcUA), etc., with UDP-GlcUA being most preferred. For UDP-GlcUA works in vivo as a GlcUA donor substrate.

The above-mentioned GalNAc donor substrate is preferably a sugar nucleotide having GalNAc and may be exemplified by adenosine diphosphate-N-acetyl-D-galactosamine (ADP-GalNAc), uridine diphosphate-N-acetyl-D-galactosamine (UDP-GalNAc), guanosine diphosphate-N-acetyl-D-galactosamine (GDP-GalNAc), cytidine diphosphate-N-acetyl-D-galactosamine (CDP-GalNAc), etc., with UDP-GalNAc being most preferred. For UDP-GalNAc works in vivo as a GalNAc donor substrate.

The “glycosytransferase 2” mentioned above is not limited in any particular way as long as it is a glycosyltransferase which functions such that a xylose residue linked to an amino acid residue has D-galactose (hereunder also referred to simply as galactose or Gal) transferred thereto by a β1,4 glycoside linkage. Such glycosyltransferase 2 plays a role at the early stage of synthesis of chondroitin/chondroitin sulfate and if it mutates, normal synthesis of chondroitin/chondroitin sulfate is believed to be prevented, leading to Paget disease of bone which can cause abnormalities in bone formation.

The “amino acid residue” as it appears in the above definition of “glycosyltransferase 2” is preferably an amino acid that forms a peptide (protein), most preferably an amino acid having linked thereto xylose which is present at the reducing terminal of a characteristic tetrasaccharide structure (GlcUAβ1-3Galβ1-3Galβ1-4Xyl: this is hereunder designated “linkage tetrasaccharide”) that exists at the linkage between a peptide (protein) and chondroitin/chondroitin sulfate in the structure of an in vivo chondroitin/chondroitin sulfate proteoglycan. The most preferred example of such amino acid is L-serine.

In short, “glycosyltransferase 2” has the activity for transferring a Gal residue from a Gal donor substrate (e.g. UDP-Gal) to a Gal acceptor containing a structure represented by formula 5 shown below to synthesize a compound containing a structure represented by formula 6 shown below (see Reference Example 5 in this specification).
Xylβ1-Ser (5)
Galβ1-4Xylβ1-Ser (6)

In above formulas 5 and 6, “Ser” represents a serine residue (which may be located in a peptide chain) having linked to its side chain a xylose residue represented by the above “Xyl”; “Xyl” represents a xylose residue linked to the side chain of a serine residue represented by said “Ser”; ‘-”, “β” and the numerals have the same meanings as defined for the above formulas 1-4.

The gene for the enzyme under consideration which comprised the nucleotide sequence (SEQ ID NO:5) was subjected to a locus search using OMIM and it was found to exist at 5q35 (see Reference Example 5 in the present specification).

Speaking of the activity such that the xylose residue linked to an amino acid residue has Gal transferred thereto by a β1,4 glycoside linkage, a Gal donor substrate that is labeled, for example, with radioactivity and a non-radiolabeled xylose linked peptide (e.g. the N-terminal sequence of bikunin having xylose linked as a side chain to a serine residue which is the 9th amino acid residue in SEQ ID NO:15; produced by Peptide Institute) may be used to perform reaction and the activity under consideration can be easily detected by subjecting the reaction product to gel filtration and analysis with an autoradiograph or scintillation counter. The above-mentioned Gal donor substrate is preferably a sugar nucleotide having Gal and may be exemplified by adenosine diphosphate-D-galactose (ADP-Gal), uridine diphosphate-D-galactose (UDP-Gal), guanosine diphosphate-D-galactose (GDP-Gal), cytidine diphosphate-D-galactose (CDP-Gal), etc., with UDP-Gal being most preferred. For UDP-Gal works in vivo as a Gal donor substrate.

Speaking of gene mutations in Paget disease of bone, it is known that in subspecies PDB2, PDB3 and PDB4, mutation has occurred at 18q22.1, 5q35 and 5q31, respectively (OMIM #602080). It is also known that gene mutations in familial Paget disease of bone are at 2q36, 10q13 and 5q35 (Am. J. Hum. Genet. 69(2001), pp. 1055-1061). In these known sites of mutation, several to ten-odd genes are coded and the positions at which the aforementioned chondroitin synthase genes are located are 5q31.1 for SEQ ID NO:1 (k3), 2q36.3 for SEQ ID NO:3 (k11) and 5q35 for SEQ ID NO:5 (β4GalT7), which are respectively in complete agreement with the positions where the above-mentioned mutations occur. Thus, all genes for those enzymes described above that participate in the synthesis of chondroitin/chondroitin sulfate overlap the sites of gene mutations in Paget disease of bone, so the probability is extremely high that the mutations of those genes will cause disorder in the biosynthesis of chondroitin/chondroitin sulfate, eventually leading to the manifestation of Paget disease of bone.

The expression which reads “associating Paget disease of bone with the mutation of a gene coding a chondroitin synthase or the amount of expression of said gene”, as used in the present specification means that a detection of a mutation in a chondroitin synthase gene or an amount of expression of that gene which is lower than the value for a normal subject is diagnosed as Paget disease of bone.

The term “gene mutation” as used in the present specification means a structural change in gene and “mutation” encompasses point mutation, inversion, deletion, insertion, duplication and translocation. A “mutant gene” means a nucleotide sequence as depicted in SEQ ID NO: 1, SEQ ID NO: 3, SEQ ID NO: 5, SEQ ID NO: 65, SEQ ID NO: 67 or SEQ ID NO: 69, provided that one or more nucleotides are replaced or deleted or that one or more nucleotides are inserted or added to said nucleotide sequence. The term “more” as used herein preferably means at least 2, more preferably at least 4, even more preferably at least 6, and most preferably at least 10.

Modes of mutation in a chondroitin synthase gene are preferably point mutation, deletion, insertion, inversion, replication and translocation, more preferably point mutation, deletion, insertion, inversion and translocation, even more preferably point mutation, deletion, insertion and translocation, and most preferably point mutation, deletion and insertion.

The term “gene expression” as used in the present specification typically means the transcription from DNA or a portion thereof to RNA or the translation from said RNA or a portion thereof to a protein. Therefore, when we say “the amount of gene expression”, we mean the amount of RNA transcribed from a gene or the amount of a protein translated from said RNA.

The amount of RNA expression can be determined, for example, by PCR analysis using the primers mentioned in Example 1 to be described later. For PCR analysis, the use of a quantitative PCR assay is preferred, and RT-PCR assay and quantitative real-time PCR assay may be given as examples for kinetics analysis. According to the present invention, these are not the only methods for quantitating RNA and Northern blot, dot blot and DNA microarrays can also be employed. If desired, nucleic acids of genes that commonly occur widely in the same tissue and the like, for example, nucleic acids that code for glyceraldehyde 3-phosphate dehydrogenase (GAPDH) and β-actin can be used as control. In addition, the amount of protein expression can be determined by, for example, ELISA and Western blot.

The detection method of the present invention can specifically be performed in the following way.

Namely, from a gene-containing specimen (which may be an epithelial tissue, a connective tissue, etc., preferably, a connective tissue, and a particularly preferred example is blood since it is extremely easy to sample and the specimen obtained is fresh enough), total RNA can be extracted and purified using, for example, an existing RNA preparing kit (e.g. RNA Extraction Kit of Amersham Biosciences K.K.) Using the extracted and purified total RNA, the amounts of K3, K11 and β4Gal-T7 expressed can be determined by RT-PCR and other methods using an existing kit (e.g. SUPERSCRIPT First-Strand Synthesis System for RT-PCR of Invitrogen Co.) Exemplary combinations of 5′-primer, 3′-primer and probe that can be employed include the combination of SEQ ID NO:56, SEQ ID NO:57 and SEQ ID NO:58 for the case of determining the amount of K3 expression, the combination of SEQ ID NO:59, SEQ ID NO:60 and SEQ ID NO:61 for the case of determining the amount of K11 expression, and the combination of SEQ ID NO:62, SEQ ID NO:63 and SEQ ID NO:64 for the case of determining the amount of βGal-T7 expression. It should be noted here that a skilled artisan can choose and prepare suitable combinations as appropriate for the specific purpose of quantification.

Then, a patient who, as the result of those quantifications, has been found to have the expression of any one of the genes K3, K11 and β4GalT7 suppressed either partly or completely can be diagnosed as a patient having high risk for Paget disease of bone. As also noted above, those quantifications can be effected by determining the amount of the expressed gene K3, K11 or β4Gal-T7 from the quantity of RNA; alternatively, an antibody against K3, K11 or β4Gal-T7 may be prepared in accordance with a conventional technique and it may be used to determine directly the quantity of the protein from K3, K11 or β4Gal-T7; yet alternatively, the activity of any one of those enzymes in a specimen may be directly measured to determine the quantity of that enzyme so that it is associated with Paget disease of bone.

More specifically, as will be mentioned in Example 2 to be described later, the ratio in the amount of expression of a chondroitin synthase gene in peripheral blood-derived mononuclear cells from a patient with Paget disease of bone and in normal peripheral blood-derived mononuclear cells may be measured by quantitative PCR to evaluate for Paget disease of bone. According to the present invention, the ratio in the amount of gene expression that can be relied upon to evaluate for Paget disease of bone is ½ or less, preferably ⅕ or less, and more preferably 1/10 or less. It should also be noted that the term “measurement” as used in the present invention covers any one of detection, amplification, quantification and semi-quantification.

According to the present invention, Paget disease of bone can be detected by confirming the presence of a mutation in a chondroitin synthase gene. To be specific, the nucleotide sequence of a chondroitin synthase gene extracted from mononuclear cells in the peripheral blood of a patient with Paget disease of bone is compared with the nucleotide sequence of the chondroitin synthase gene extracted from mononuclear cells in normal peripheral blood and the detection of any mutation can be identified as indicative of Paget disease of bone. To be more specific, as will be set forth in Example 1 to be described later, primers prepared appropriately on the basis of the nucleotide sequences of K3, K11 and β4Gal-T7 described in the Sequence Listing as accompanying the present specification are used to amplify the gene encoding K3, K11 or β4Gal-T7 directly from genomic DNA by a suitable technique such as PCR assay and its nucleotide sequence is analyzed in accordance with a known nucleotide sequencing method, with the presence of any mutation in one of those genes being held as indicative of Paget disease of bone. The number of mutant nucleotides that can be considered to be indicative of Paget disease of bone is at least 1, preferably at least 4, more preferably at least 6, and most preferably at least 10.

Modes of mutation in the chondroitin synthase genes are preferably point mutation, deletion, insertion, inversion, replication and translocation, more preferably point mutation, deletion, insertion, inversion and translocation, even more preferably point mutation, deletion, insertion and translocation, and most preferably point mutation, deletion and insertion.

Now that it has become clear according to the present invention that K3, K11 and β4Gal-T7 are associated with Paget disease of bone, a knockout animal which shows the pathology of Paget disease of bone can be prepared by suppressing the expression of those genes either partly or completely in a laboratory animal. Preferred knockout animals are mammals including mouse, rat, rabbit, dog, cat and monkey, with mouse and rat being most preferred since the techniques of preparing knockout animals from them have been established to some extent; however, those are not the sole examples of the knockout animals that can be prepared in the present invention.

For instance, knockout mice (pathological model mice) that show the pathology of Paget disease of bone can be prepared in accordance with the descriptions in “Latest Technology of Gene Targeting” (ed. by T. Yagi, Yodosha Co., LTD.) and “Gene Targeting” (translated and supervised by T. Noda, Medical Sciences International, LTD.)

Suppose, for instance, that a mouse gene to be knocked out is K3 (mK3: SEQ ID NO:65), K11 (mK11: SEQ ID NO:67) or β4Gal-T7 (mβ4Gal-T7: SEQ ID NO:69). A linear targeting vector (80 μg) with an insert of chromosomal fragment (ca. 10 kb) centering on an exon that contains a domain for activating that gene (a fragment containing the third exon (1560 bp) in the ORF portion of mK3; a fragment containing 4598 bp corresponding to the full length of ORF in mK11; and a fragment containing the second to sixth exons of ORF in β4Gal-T7) is introduced into ES cells (derived from E14/129Sv mouse) by a known method such as electroporation; G418 resistant colonies are selected, transferred to a 24-well plate and cultivated; after storing some of the cells in a frozen state, DNA is extracted from the remaining ES cells and about 120 colonies of clones that are found to have undergone recombination are selected by a known nucleotide sequence analyzing method; further, Southern blotting and other suitable methods are employed to confirm that recombination has taken place as intended; finally, about 10 recombinant clones are selected and ES cells from about two of them are injected into blastocytes of C57BL/6 mouse; the mouse embryo into which the ES cells were thusly injected is transplanted into the womb of a surrogate mouse which will then give birth to a chimera mouse; a hetero knockout mouse can be obtained by subjecting the chimera mouse to germ transmission.

A knockout mouse can also be prepared by a method for suppressing gene expression, such as the small interfering RNA assay (T. R. Brummelkamp et al., Science, 296, 550-553 (2002)).

More specifically, as set forth in Example 3 to be described later, a knockout mouse can be prepared by first transferring a chondroitin synthase gene into ES cells and then using clones that have undergone the intended homologous recombination to establish a mouse lineage in a reproductive cell line.

### EXAMPLES

On the pages that follow, the present invention is described more specifically by means of Examples.

### Reference Example 1

### Preparation of Chondroitin Sulfate Synthase (K3)

(1) Cloning of cDNA and Constructing an Expression Vector

BLAST search was performed using as a query the amino acid sequence of β1,4-galactose transferase (β4Gal-T) (the amino acid sequence encoded by GenBank accession No. D29805). As the result, Expression Sequence Tag (EST: GenBank accession No. AC0004219) was discovered. Since this sequence was incomplete, ORF was investigated from a genomic database by means of GenScan (Stanford University, USA). As the result, the nucleotide sequence depicted in SEQ ID NO: 1 (encoding the amino acid sequence of SEQ ID NO: 2) was discovered. The gene comprising the nucleotide sequence depicted in SEQ ID NO: 1 is at least expressed in the human brain and this was verified by RT-PCR assay using Marathon-Ready cDNA (CLONTECH Laboratories, Inc.) as a template (as PCR primers, the combinations of 5′ primer and 3′ primer according to SEQ ID NO: 9 and SEQ ID NO: 10, as well as SEQ ID NO: 11 and SEQ ID NO: 12 were used). In order to clone a soluble region of the gene product of interest other than a region including the transmembrane region (the region to be excluded comprising amino acid Nos. 1-129 in SEQ ID NO: 2), PCR was performed according to a conventional technique using the two primers of SEQ ID NO: 7 and SEQ ID NO: 8. The template cDNA used was Marathon-Ready cDNA human brain (CLONTECH Laboratories, Inc.) The amplified band of about 2.3 kb was digested with HindIII and XbaI according to a conventional technique and inserted between HindIII and XbaI sites of a mammalian cell expression vector pFLAG-CMV-1 (Sigma) according to a conventional technique, thereby making an expression vector (K3-FLAG-CMV1). Nucleotide sequencing of the obtained expression vector showed the insertion of a DNA fragment comprising a nucleotide sequence of nucleotide Nos. 388-2649 in the nucleotide sequence depicted in SEQ ID NO: 1. The genomic position of DNA having that nucleotide sequence was identified by using OMIM and it was found to exist at 5q31.1.

(2) Preparing K3

K3-FLAG-CMV1 (15 μg) was set on TransFast (Promega Corp.) which was operated according to the protocol to transfer the gene into COS7 cells that had been cultivated on 100 mm culture dishes to produce 70% confluent cultures. After 3-day cultivation, the supernatant was recovered and passed through a 0.22 μm filter; thereafter, 100 μl of Anti-FLAG M2-Agarose Affinity Gel (Sigma) was added to 10 ml of the supernatant and mixing by inverting of the tube was performed overnight at 4° C. After the reaction, the gel was washed three times with 50 mM Tris-HCl (pH 7.4)/20% glycerol and the unwanted wash solution was removed with a syringe fitted with a 27 G needle. The gel was suspended in 50 mmol/L Tris-HCl at pH 7.4 (20 glycerol, 10 mmol/L phenylmethyl sulfonyl fluoride, 1 μg/ml leupeptin and 1 μg/ml pepstatin) to make 50 (v/v); after centrifugation, the supernatant was removed to prepare an enzyme adsorbed gel suspension.

### Reference Example 2

### Extension of Chondroitin Building Blocks Using K3

(1) Preparation of Chondroitin/Chondroitin Sulfate Odd-Numbered Saccharides

Chondroitin (shark cartilage derived chondroitin sulfate as chemically desulfurylated: product of Seikagaku Corporation) and chondroitin sulfate (as derived from shark cartilage: product of Seikagaku Corporation: also called chondroitin sulfate C) were subjected to limited digestion with bovine testis hyaluronidase (Sigma) and thereafter the reaction solution was held at 100° C. for 10 minutes until the enzyme was deactivated thermally. The reaction solution was loaded on a Superdex 30 column (60×1.6 cm: product of Amersham Biosciences K.K.; chromatographic conditions: mobile phase, 0.2 mol/L NH₄HCO₃; flow rate, 2 ml/min) and the effluent was fractionated for each 2 ml with monitoring at an absorbance of 225 nm, followed by pooling of fractions equivalent to decasaccharide. Those fractions were desalted by PD10 column (Amersham Biosciences K.K.) and after uronic acid was quantitated by the carbazole sulfate method according to a conventional technique, the fractions were freeze-dried. The freeze-dried product was dissolved in distilled water to give 1 mM, thereby preparing even-numbered oligosaccharide samples (chondroitin derived decasaccharide is hereunder designated CH10, and chondroitin sulfate derived decasaccharide as CS10).

To 50 mmol/L MES buffer solution (pH 6.5) containing 10 nmol/L MnCl₂ and 171 μmol/L ATP sodium salt, 10 μl of the enzyme adsorbed gel suspension, 1 nmol of a test substance (chondroitin (CHEL), chondroitin sulfate (CSEL), CH10 or CS10) and 0.036 nmol of [³H]UDP-GalNAc were added to make a total volume of 30 μl. Enzymatic reaction was carried out at 37° C. for 1 hour and thereafter the reaction solution was kept at 100° C. for 1 minute until the enzyme was deactivated for quenching the reaction.

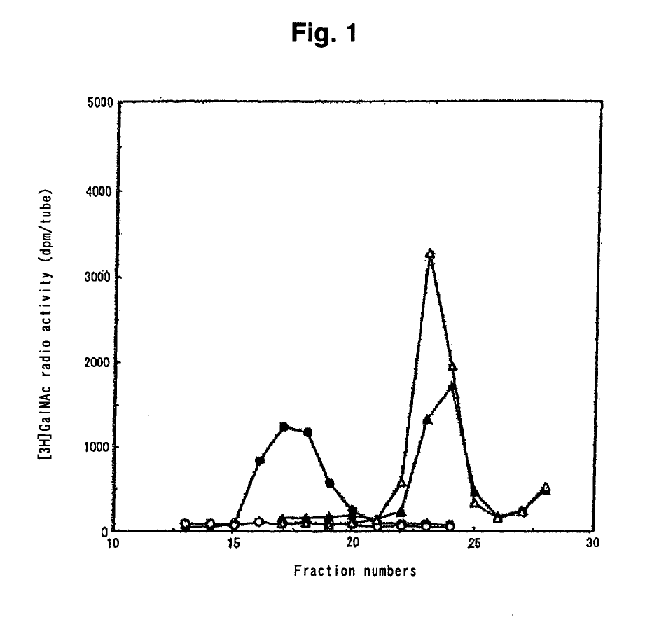

Each of the reaction solutions was passed through a microfilter with a pore size of 0.22 μm (Millipore Corp.); thereafter, it was separated by a Superdex peptide column (30×1.0 cm: product of Amersham Biosciences K.K.; chromatographic conditions: mobile phase, 0.2 mol/L NaCl; flow rate, 1.0 ml/min) and the effluent was recovered at 0.5 ml fractions, followed by radioactivity measurement with a scintillation counter (FIG. 1). As the result, strong GalNAc transfer activity was observed when CHEL (fractions 17-18), CH10 (fraction 23) and CS10 (fraction 23) were used as a GalNAc acceptor substrate but no GalNAc transfer activity was observed with CSEL (fraction 16). Fraction 23 as each of the reaction products from CH10 and CS10 showed a molecular weight at which undecasaccharide was eluted. The undecasaccharide obtained from CH10 was designated CH11(K3) and the one obtained from CS10 designated as CS11(K3).

Fractions 21-25 of CS11(K3) were recovered, pooled and desalted with a PD10 column. The thus obtained sample was divided into two equal portions and freeze-dried. One of the two halved portions was dissolved in 100 μl of 0.1 mol/L Tris-HCl buffer solution (pH 7.4) containing 30 mM sodium acetate; the resulting solution was designated CS11(K3)A. The other portion was digested with chondroitinase ACII (100 mU of chondroitinase ACII (Seikagaku Corporation) was dissolved in 100 μl of CS11(K3) fraction, digested enzymatically at 37° C. for 10 hours, and heated to deactivate the enzyme; the resulting product was designated CS11(K3)B).

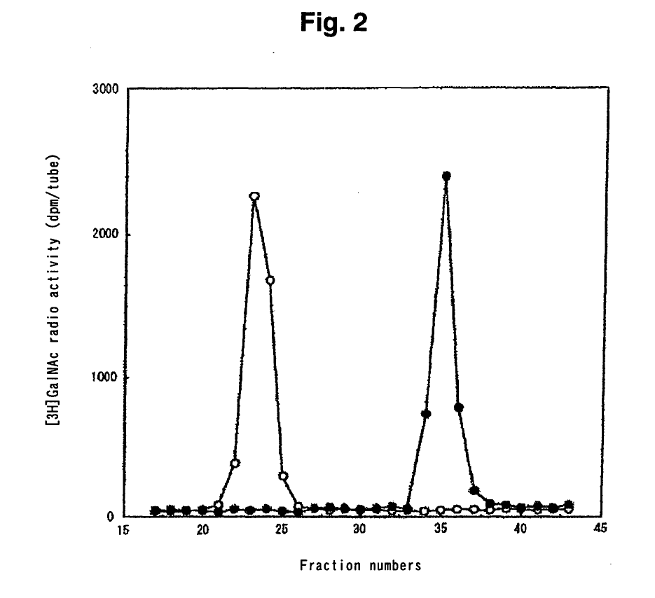

CS11(k3)A and CS11(K3)B were passed through a microfilter with a pore size of 0.22 μm (Millipore Corp.); thereafter, they were separated by a Superdex peptide column (30×10 mm: product Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter to give a radioactivity peak shift to a monosaccharaide fraction in CS11(K3)B (FIG. 2). From this result, it became clear that K3 transferred GalNAc to GlcUA at the non-reducing terminal of the chondroitin sulfate derived decasaccharide via β1,4 linkage to prepare an undecasaccharide.

(2) Preparation of Chondroitin/Chondroitin Sulfate Even-Numbered Saccharides

Chondroitin (shark cartilage derived chondroitin sulfate as chemically desulfurylated: product of Seikagaku Corporation) and chondroitin sulfate (as derived from shark cartilage: product of Seikagaku Corporation: also called chondroitin sulfate C) were subjected to limited digestion with bovine testis hyaluronidase (Sigma) and thereafter the reaction solution was held at 100° C. for 10 minutes until the enzyme was deactivated thermally. The reaction solution was centrifuged at 10,000×g for 10 minutes and the recovered supernatant was further digested with bovine liver derived β glucuronidase (Sigma). The enzymatic reaction was quenched by keeping the reaction solution at 100° C. for 10 minutes. The reaction solution was then loaded on a Superdex 30 column (60×1.6 cm: product of Amersham Biosciences K.K.; chromatographic conditions: mobile phase, 0.2 mol/L NH₄HCO₃; flow rate, 2 ml/min) and the effluent was fractionated for each 2 ml with monitoring at an absorbance of 225 nm, followed by pooling of fractions equivalent to undecasaccharide. Those fractions were desalted by PD10 column (Amersham Biosciences K.K.) and after uronic acid was quantitated by the carbazole sulfate method according to a conventional technique, the fractions were freeze-dried. The freeze-dried product was dissolved in distilled water to give 1 mmol/L, thereby preparing odd-numbered oligosaccharide samples (chondroitin derived undecasaccharide is hereunder designated CH11, and chondroitin sulfate derived decasaccharide as CS11).

In addition, chondroitin (shark cartilage derived chondroitin sulfate as chemically desulfurylated: product of Seikagaku Corporation) and chondroitin sulfate (as derived from shark cartilage: product of Seikagaku Corporation: also called chondroitin sulfate C) were digested with bovine liver derived β glucuronidase (Sigma) to prepare samples (which are respectively degignated CHOL and CSOL).

To 50 mmol/L acetate buffer solution (pH 5.6) containing 10 mmol/L MnCl₂, 10 μl of the enzyme adsorbed gel suspension, 1 nmol of a test substance (CHOL, CSOL, CH11 or CS11) and 0.432 nmol of [¹⁴C]UDP-GlcUA were added to make a total volume of 30 μl. Enzymatic reaction was carried out at 37° C. for 1 hour and thereafter the reaction solution was kept at 100° C. for 1 minute until the enzyme was deactivated for quenching the reaction.

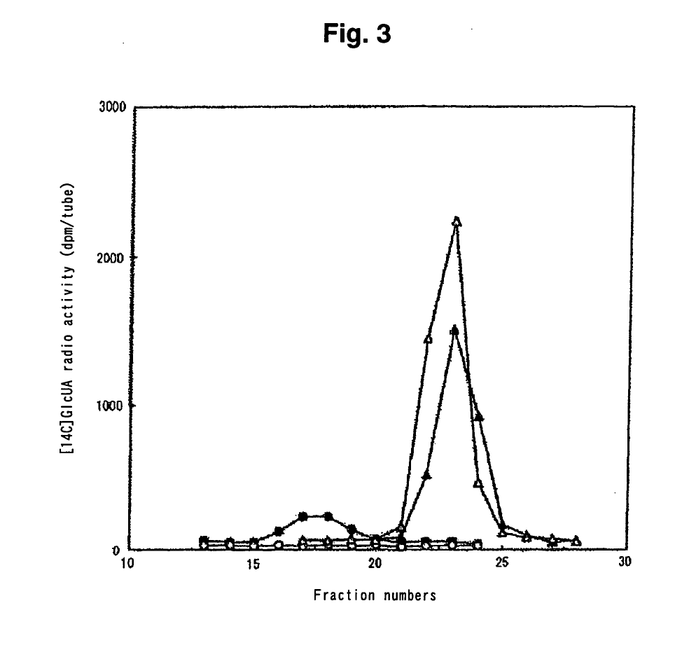

Each of the reaction solutions was passed through a microfilter with a pore size of 0.22 μm (Millipore Corp.); thereafter, it was separated by a Superdex peptide column (30×1.0 cm: product of Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter (FIG. 3). As the result, strong GlcUA transfer activity was observed when CHOL (fractions 17-18), CH11 (fraction 23) and CS11 (fraction 23) were used as a GlcUA acceptor substrate but no GlcUA transfer activity was observed with CSOL (fraction 16). Fractions 22-23 as the reaction products from CH11 and CS11 showed molecular weights at which dodecasaccharide was eluted. The dodecasaccharide obtained from CH11 was designated CH12(K3) and the one obtained from CS11 designated as CS12(K3).

Fractions 21-25 of CS12(K3) were recovered, pooled and desalted with a PD10 column. The thus obtained sample was divided into two equal portions and freeze-dried. One of the two halved portions was dissolved in 100 μl of 0.1 mol/L Tris-HCl buffer solution (pH 7.4) containing 30 mmol/L sodium acetate; the resulting solution was designated CS12(K3)A. The other portion was digested with chondroitinase ACII (100 munits of chondroitinase ACII (Seikagaku Corporation) was dissolved in 100 μl of CS11(K3) fraction, digested enzymatically at 37° C. for 10 hours, and heated to deactivate the enzyme; the resulting product was designated CS12(K3)B).

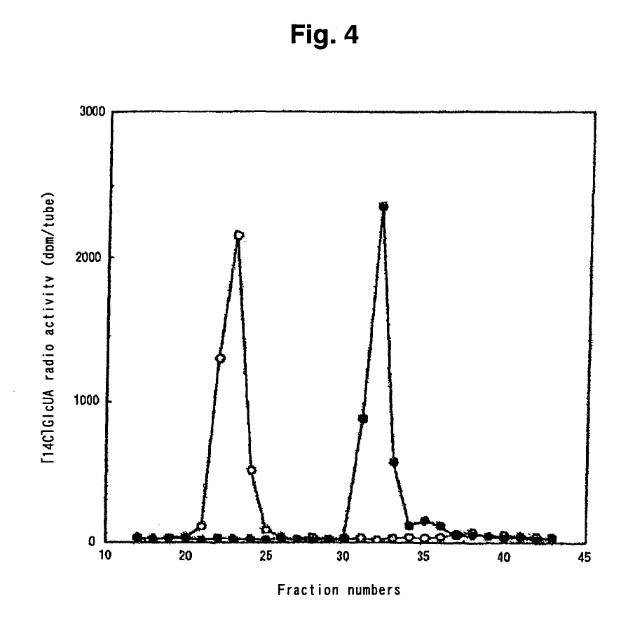

CS12(K3)A and CS12(K3)B were passed through a microfilter with a pore size of 0.22 μm (Millipore Corp.); thereafter, they were separated by a Superdex peptide column (30×1.0 cm: product of Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter to give a radioactivity peak shift to a disaccharaide fraction in CS12(K3)B (FIG. 4). From this result, it became clear that K3 transferred GlcUA to the chondroitin sulfate derived undecasaccharide via β1,3 linkage to prepare a dodecasaccharide.

### Reference Example 3

### Preparation of Chondroitin Sulfate Synthase (K11)

(1) Cloning of cDNA and Constructing an Expression Vector

BLAST search was performed using as a query the amino acid sequence of chondroitin sulfate glucuronic acid transferase (CSGlcA-T) (the amino acid sequence encoded by GenBank accession No. AB037823). As the result, EST (GenBank accession NM_{—}018590) was discovered. Since this sequence was incomplete, ORF was investigated from a genomic database by means of GenScan (Stanford University, USA). As the result, the nucleotide sequence depicted in SEQ ID NO: 3 (encoding the amino acid sequence of SEQ ID NO: 4) was discovered. The gene comprising the nucleotide sequence depicted in SEQ ID NO: 3 is at least expressed in the human brain and this was verified by RT-PCR assay using Marathon-Ready cDNA (CLONTECH Laboratories, Inc.) as a template. In order to clone a soluble region of the gene of interest other than a region including the transmembrane region (the region to be excluded comprising amino acid Nos. 1-96 in SEQ ID NO: 4), PCR was performed according to a conventional technique using the two primers of SEQ ID NO: 13 and SEQ ID NO: 14. The template cDNA used was Marathon-Ready cDNA human brain (CLONTECH Laboratories, Inc.) The amplified band of about 2 kb was digested with EcoRI and BamHI according to a conventional technique and inserted between EcoRI and BamHI sites of a mammalian cell expression vector pFLAG-CMV-1 (Sigma) according to a conventional technique, thereby making K11-FLAG-CMV1. Nucleotide sequencing of the obtained vector showed the insertion of a DNA fragment comprising a nucleotide sequence of nucleotide Nos. 287-2328 in the nucleotide sequence depicted in SEQ ID NO: 3. The genomic position of DNA having that nucleotide sequence was identified by using OMIM and it was found to exist at 2q36.3.

(2) Preparing K11

K11-FLAG-CMV1 (15 mg) was set on TransFast (Promega Corp.) which was operated according to the protocol to transfer the gene into COS7 cells that had been cultivated on 100 mm culture dishes to produce 70% confluent cultures. After 3-day cultivation, the supernatant was recovered and passed through a 0.22 μm filter; thereafter, 100 μl of Anti-FLAG M2-Agarose Affinity Gel (Sigma) was added to 10 ml of the supernatant and mixing by inverting of the tube was performed overnight at 4° C. After the reaction, the gel was washed three times with 50 mmol/L Tris-HCl at pH 7.4 (20 glycerol) and the unwanted wash solution was removed with a syringe fitted with a 27 G needle. The gel was suspended in 50 mmol/L Tris-HCl at pH 7.4 (20% glycerol, 10 mmol/L phenylmethyl sulfonyl fluoride, 1 μg/ml leupeptin and 1 μg/ml pepstatin) to make 50 (v/v); after centrifugation, the supernatant was removed to prepare an enzyme adsorbed gel suspension.

### Reference Example 4

### Extension of Chondroitin Building Blocks Using K11

(1) Preparation of Chondroitin/Chondroitin Sulfate Odd-Numbered Saccharides

To 50 mmol/L MES buffer solution (pH 6.5) containing 10 nmol/L MnCl₂ and 171 μmol/L ATP sodium salt, 10 μl of the enzyme adsorbed gel suspension, 1 nmol of a test substance (CHEL, CSEL, CH10 or CS10) and 0.036 nmol of [³H]UDP-GalNAc were added to make a total volume of 30 μl. Enzymatic reaction was carried out at 37° C. for 1 hour and thereafter the reaction solution was kept at 100° C. for 1 minute until the enzyme was deactivated for quenching the reaction.

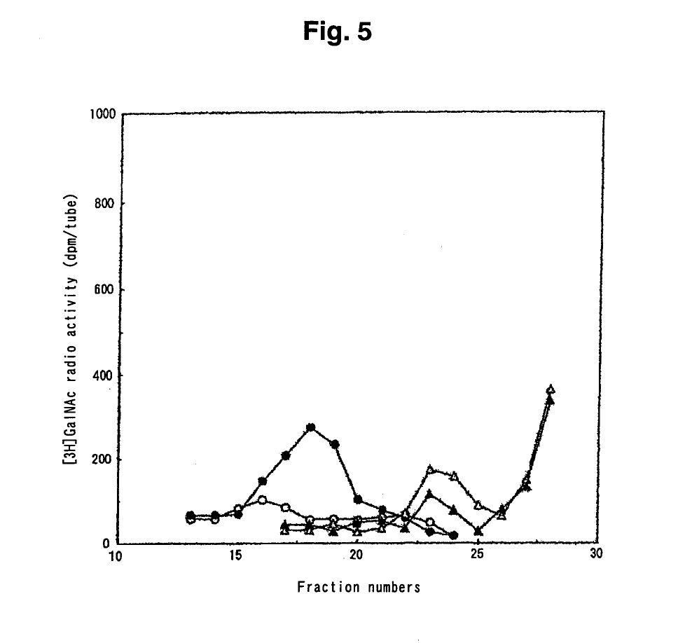

Each of the reaction solutions was passed through a microfilter with a pore size of 0.22 μm (Millipore Corp.); thereafter, it was separated by a Superdex peptide column (30×1.0 cm: product of Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter (FIG. 5). As the result, strong GalNAc transfer activity was observed when CHEL (fraction 18), CH10 (fraction 23) and CS10 (fraction 23) were used as a GalNAc acceptor substrate and weak GalNAc transfer activity was observed with CSEL (fraction 16). Fractions 22-23 as the reaction products from CH10 and CS10 showed molecular weights at which undecasaccharide was eluted. The undecasaccharide obtained from CH10 was designated CH11(K11) and the one obtained from CS10 designated as CS11(K11).

Fractions 21-25 of CS11(K11) were recovered, pooled and desalted with a PD10 column. The thus obtained sample was divided into two equal portions and freeze-dried. One of the two halved portions was dissolved in 100 μl of 0.1 mol/L Tris-HCl buffer solution (pH 7.4) containing 30 mM sodium acetate; the resulting solution was designated CS11(K11)A. The other portion was digested with chondroitinase ACII (100 munits of chondroitinase ACII (Seikagaku Corporation) was dissolved in 100 μl of CS11(K11) fraction, digested with an enzyme at 37° C. for 10 hours, and heated to deactivate the enzyme; the resulting product was designated CS11(K11)B).

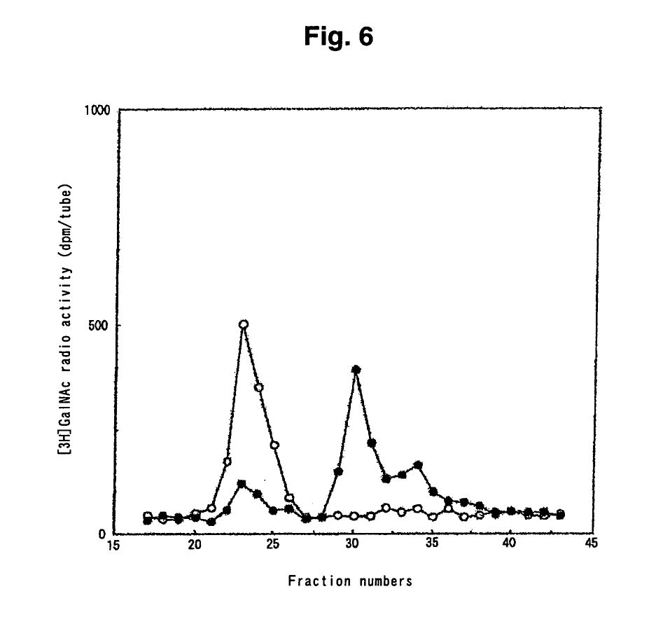

CS11(K11)A and CS11(K11)B were passed through a microfilter with a pore size of 0.22 μm (Millipore Corp.); thereafter, they were separated by a Superdex peptide column (30×10 mm: product of Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter to give a radioactivity peak shift to a trisaccharaide fraction in CS11(K11)B (FIG. 6). From this result, it became clear that K11 transferred GalNAc to GlcUA at the non-reducing terminal of the chondroitin sulfate derived decasaccharide via β1,4 linkage to prepare an undecasaccharide.

(2) Preparation of Chondroitin/Chondroitin Sulfate Even-Numbered Saccharides

To 50 mmol/L acetate buffer solution (pH 5.6) containing 10 nmmol/L MnCl₂, 10 μl of the enzyme adsorbed gel suspension, 1 nmol of a test substance (CHOL, CSOL, CH11 or CS11) and 0.432 nmol of [¹⁴C]UDP-GlcUA were added to make a total volume of 30 μl. Enzymatic reaction was carried out at 37° C. for 1 hour and thereafter the reaction solution was kept at 100° C. for 1 minute until the enzyme was deactivated for quenching the reaction.

Each of the reaction solutions was passed through a microfilter with a pore size of 0.22 μm (Millipore Corp.); thereafter, it was separated by a Superdex peptide column (30×1.0 cm: product of Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter (FIG. 3). As the result, strong GlcUA transfer activity was observed when CHOL (fractions 18), CH11 (fraction 23) and CS11 (fraction 22) were used as a GlcUA acceptor substrate but no GlcUA transfer activity was observed with CSOL. Fractions 22-23 as the reaction products from CH11 and CS11 showed molecular weights at which dodecasaccharide was eluted. The dodecasaccharide obtained from CH11 was designated CH12(K11) and the one obtained from CS11 designated as CS12(K11).

Fractions 21-25 of CS12(K11) were recovered, pooled and desalted with a PD10 column. The thus obtained sample was divided into two equal portions and freeze-dried. One of the two halved portions was dissolved in 100 μl of 0.1 mol/L Tris-HCl buffer solution (pH 7.4) containing 30 mmol/L sodium acetate; the resulting solution was designated CS12(K11)A. The other portion was digested with chondroitinase ACII (100 munits of chondroitinase ACII (Seikagaku Corporation) was dissolved in 100 μl of CS11(K11) fraction, digested enzymatically at 37° C. for 10 hours, and heated to deactivate the enzyme; the resulting product was designated CS12(K11)B).

CS12(K11)A and CS12(K11)B were passed through a microfilter with a pore size of 0.22 μm (Millipore Corp.); thereafter, they were separated by a Superdex peptide column (30×1.0 cm: product of Amersham Biosciences K.K.; chromatographic conditions: mobile phase, 0.2 mol/L NaCl; flow rate, 0.5 ml/min) and the effluent was recovered at 0.5 ml fractions, followed by radioactivity measurement with a scintillation counter to give a radioactivity peak shift to a disaccharaide fraction in CS12(K11)B (FIG. 4). From this result, it became clear that the enzyme of the present invention could transfer GlcUA to the chondroitin sulfate derived undecasaccharide via β1,3 linkage to prepare a dodecasaccharide.

### Reference Example 5

### Preparation of Chondroitin Sulfate Synthase (β4Gal-T7) and Verification of its Activity

Cloning of β4Gal-T7 was performed according to the method of Almeida et al. (EXPERIMENTAL PROCEDURES in J. Biol. Chem., 274, 37, 26165-26171 (1999)). The nucleotide sequence of the clones obtained was verified by a conventional technique and it was found to comprise the nucleotide sequence depicted in SEQ ID NO: 5. The genomic position of DNA having that nucleotide sequence was identified by using OMIM and it was found to exist at 5q35.

Further, in accordance with the method of Gotoh et al. (J. Biol. Chem., 277, 41, 38189-38196 (2002)), β4Gal-T7 was expressed and there was prepared a suspension of an affinity carrier having the β4Gal-T7 linked thereto; thereafter, 10 μl of the gel, 6.25 mg of a acceptor substrate (xylose linked peptide [the N-terminal sequence of bikunin having xylose linked as a side chain to serine which was the amino acid residue at 9-position of SEQ ID NO: 15]: product of Peptide Institute), 171 μmol of adenosine triphosphate (ATP) sodium salt and 0.036 mol of [³H]UDP-Gal were added to 50 mmol of MES buffer solution (pH 6.5) containing 10 nmol/L MnCl₂ to make a total volume of 30 μl and reaction was carried out at 37° C. for 1 hour. Thereafter, the reaction solution was kept at 100° C. for 1 minute until the enzyme was deactivated for quenching the reaction.

The reaction solution was passed through a microfilter with a pore size of 0.22 μm (Millipore Corp.); thereafter, it was separated by a Superdex peptide column (30×1.0 cm: product of Amersham Biosciences K.K.; chromatographic conditions: mobile phase, 0.2 mol/L NaCl; flow rate, 1.0 ml/min) and the effluent was recovered at 0.5 ml fractions, followed by radioactivity measurement with a scintillation counter. As the result, strong radioactivity was observed in a polymer fraction other than the eluted fraction of [³H]UDP-Gal. This supported the linking by β4Gal-T7 of galactose to the acceptor substrate.

### Example 1

### Extraction of DNA From Blood Sample and Analysis of its Nucleotide Sequence

DNA was extracted from 1 ml of blood using GFX Genomic Blood DNA Purification Kit (Amersham Biosciences K.K.) Of the 14-16 μg of DNA obtained, 250 ng was used as a template to amplify the exons in the genes of chondroitin sulfate synthases (K3, K11, β4Gal-T7) with the aid of various primers (see Table 1 below). Using the thus obtained fragments as templates, the nucleotide sequences of the genes were analyzed by a conventional technique and compared with those of the genes of normal types of the respective chondroitin synthases; in this way, one could easily check for the presence of mutations and single nucleotide polymorphisms.

TABLE 1

| | For exon amplification | | For nucleotide |
|---|---|---|---|
| | 5′ primer | 3′ primer | sequence analysis |
| K3 (Exon 1) | SEQ ID | SEQ ID | SEQ ID NO: 16, SEQ ID NO: 17, |
| | NO: 16 | NO: 17 | SEQ ID NO: 18, SEQ ID NO: 19 |
| K3 (Exon 2) | SEQ ID | SEQ ID | SEQ ID NO: 20, SEQ ID NO: 21 |
| | NO: 20 | NO: 21 | |
| K3 (Exon 3) | SEQ ID | SEQ ID | SEQ ID NO: 22, SEQ ID NO: 23, |
| | NO: 22 | NO: 23 | SEQ ID NO: 24, SEQ ID NO: 25, |
| | | | SEQ ID NO: 26, SEQ ID NO: 27, |
| | | | SEQ ID NO: 28, SEQ ID NO: 29, |
| | | | SEQ ID NO: 30, SEQ ID NO: 31 |
| K11 | SEQ ID | SEQ ID | SEQ ID NO: 32, SEQ ID NO: 33 |
| (Exon 1) | NO: 32 | NO: 33 | |
| K11 | SEQ ID | SEQ ID | SEQ ID NO: 34, SEQ ID NO: 35 |
| (Exon 2) | NO: 34 | NO: 35 | |
| K11 | SEQ ID | SEQ ID | SEQ ID NO: 36, SEQ ID NO: 37 |
| (Exon 3) | NO: 36 | NO: 37 | |
| K11 | SEQ ID | SEQ ID | SEQ ID NO: 38, SEQ ID NO: 39, |
| (Exon 4) | NO: 38 | NO: 39 | SEQ ID NO: 40, SEQ ID NO: 41, |
| | | | SEQ ID NO: 42, SEQ ID NO: 43 |
| β4Gal-T7 | SEQ ID | SEQ ID | SEQ ID NO: 44, SEQ ID NO: 45 |
| (Exon 1) | NO: 44 | NO: 45 | |
| β4Gal-T7 | SEQ ID | SEQ ID | SEQ ID NO: 46, SEQ ID NO: 47 |
| (Exon 2) | NO: 46 | NO: 47 | |
| β4Gal-T7 | SEQ ID | SEQ ID | SEQ ID NO: 48, SEQ ID NO: 49 |
| (Exon 3) | NO: 48 | NO: 49 | |
| β4Gal-T7 | SEQ ID | SEQ ID | SEQ ID NO: 50, SEQ ID NO: 1 |
| (Exon 4) | NO: 50 | NO: 51 | |
| β4Gal-T7 | SEQ ID | SEQ ID | SEQ ID NO: 52, SEQ ID NO: 53 |
| (Exon 5) | NO: 52 | NO: 53 | |
| β4Gal-T7 | SEQ ID | SEQ ID | SEQ ID NO: 54, SEQ ID NO: 55 |
| (Exon 6) | NO: 54 | NO: 55 | |

### Example 2

### Extraction of RNA From Blood Sample and Analysis of the Amount Expressed

A blood sample (10 ml) was centrifuged at 3,000 rpm for 10 minutes and the intermediate layer was separated off. Further centrifugation was performed using Lympho Prep (Nycomed Inc.) and the intermediate layer was separated off. By these procedures, 9×10⁷ peripheral blood-derived mononuclear cells (PBMC) were obtained. From all quantities of PBMC, whole RNA can be prepared using RNA Extraction Kit (Amersham Biosciences K.K.) From 10 ng of the whole RNA, 1st strand cDNA was synthesized by SUPERSCRIPT First-Stand Synthesis System for RT-PCR (Invitrogen Co.) Using this cDNA as a template, real-time PCR assay was performed to determine the amounts of various genes expressed. The primers and probes used are shown in

TABLE 2

| | 5′ Primer | 3′ Primer | Probe |
|---|---|---|---|
| K3 | SEQ ID NO: 56 | SEQ ID NO: 57 | SEQ ID NO: 58 |
| K11 | SEQ ID NO: 59 | SEQ ID NO: 60 | SEQ ID NO: 61 |
| β4Gal-T7 | SEQ ID NO: 62 | SEQ ID NO: 63 | SEQ ID NO: 64 |

### Example 3

### Preparation of Pathogenic Model Mouse

Knockout mice were obtained by the following procedure using a mouse gene K3 (mK3: SEQ ID NO:65), K11 (mK11: SEQ ID NO:67) or β4Gal-T7 (mβ4Gal-T7: SEQ ID NO:69). A linear targeting vector (80 μg) with an insert of chromosomal fragment (ca. 10 kb) centering on an exon that contained a domain for activating that gene (a fragment containing the third exon (1560 bp) in the ORF portion in the case of mK3; a fragment containing 4598 bp corresponding to the full length of ORF in mK11; and a fragment containing the second to sixth exons of ORF in β4Gal-T7) was introduced into ES cells (derived from E14/129Sv mouse) by electroporation; G418 resistant colonies were selected; the G418 resistant colonies were transferred to a 24-well plate and cultivated; after storing some of the cells in a frozen state, DNA was extracted from the remaining ES cells and about 120 colonies of clones that were found to have undergone recombination were selected by PCR; further, Southern blotting was employed to confirm that recombination had taken place as intended; finally, about 10 recombinant clones were selected and ES cells from about two of them were injected into blastocytes of C57BL/6 mouse; the mouse embryo into which the ES cells were thusly injected was transplanted into the womb of a surrogate mouse which then gave birth to a chimera mouse; by subsequent germ transmission, a hetero knockout mouse was obtained.

### REFERENCE DOCUMENTS

1. J. Bone Miner Res., 11(1996), pp. 1602-1607
2. Endocr. Metab. Clin. North. Am., 24(1995), pp. 437-450
3. Am. J. Hum. Genet., 69(2001), pp. 1055-1061

### INDUSTRIAL APPLICABILITY

According to the present invention, there are provided a new method of detecting Paget disease of bone and a pathogenic animal with Paget disease of bone.

1. A method for detecting Paget disease of bone by associating Paget disease of bone with the mutation of a gene coding a chondroitin synthase gene or the amount of expression of said gene.

2. The method for detecting Paget disease of bone according to claim 1, wherein the chondroitin synthase is a glycosyltransferase having activity for transferring a D-glucuronic acid residue or an N-acetyl-D-galactosamine residue to the saccharide residue at the non-reducing terminal of chondroitin.

3. The method for detecting Paget disease of bone according to claim 1, wherein the chondroitin synthase is a glycosyltransferase by means of which a xylose residue linked to an amino acid residue has D-galactose linked thereto by a β1,4-glycoside linkage.

4. The method for detecting Paget disease of bone according to claim 1, wherein the chondroitin synthase gene is a gene comprising the nucleotide sequence depicted in any one of SEQ ID NO: 1, SEQ ID NO: 3, SEQ ID NO: 5, SEQ ID NO: 65, SEQ ID NO: 67 or SEQ ID NO: 69.

5. A knockout animal wherein a glycosyltransferase gene comprising the amino acid sequence depicted in any one of SEQ ID NO: 2, SEQ ID NO: 4, SEQ ID NO: 6, SEQ ID NO: 66, SEQ ID NO: 68 or SEQ ID NO: 70 or any one of the amino acid sequences homologous thereto is partly or completely suppressed in expression.
